(19) United States
(12) Patent Application Publication
(10) Pub. No.: US 2026/0278981 A1
(43) Pub. Date: Sep. 17, 2026

(54) MACHINE-LEARNING BASED METHOD FOR RECOMMENDING EYEWEAR FOR INDIVIDUALS USING SELF CORRECTION

(71) Applicant: SITREP HEALTH, LLC, Livermore, CA (US)

(72) Inventors: Ryan WILKERSON, Livermore, CA (US); Ian SIDDIQI, Valley Village, CA (US)

(73) Assignee: SITREP HEALTH, LLC, Livermore, CA (US)

(21) Appl. No.: 19/266,069

(22) Filed: Jul. 10, 2025

Related U.S. Application Data
(60) Provisional application No. 63/773,281, filed on Mar. 17, 2025.

(51) Int. Cl. G06V 10/24; G06V 10/75; G06V 20/40; G06V 40/16; G16H 20/00

(52) U.S. Cl. CPC G06V 10/245; G06V 10/751; G06V 20/46; G06V 40/171; G16H 20/00

(57) ABSTRACT

Disclosed herein are systems and methods for a machine-learning based method for correcting facial landmark positions. The method includes performing a facial scan by capturing an image of user’s face and generating video frames from the captured image; extracting facial landmark estimates corresponding to the face of a user from each frame of video frames using a pre-trained facial landmark detection MLM; storing each of the facial landmark estimates as a vector in a time-sequenced matrix; validating the facial landmark estimates by comparing consecutive video frames from the plurality of video frames to detect facial landmark drift using a pre-trained self-correction MLM; based on a determination that the pre-trained machine learning model detects facial landmark drift, generating recalibrated facial landmark estimates by performing recalibration on the consecutive frames; and recommending eyewear frames based on the validated facial landmark estimates or the recalibrated facial landmark estimates.

### CROSS REFERENCE TO RELATED APPLICATIONS

This application claims priority to U.S. Patent Provisional Application No. 63/773,281, filed Mar. 17, 2025, the entire contents of which are hereby incorporated by reference.

### FIELD OF TECHNOLOGY

The present disclosure relates to the field of machine learning, and, more specifically, to systems and methods for recommending eyewear using self-correction.

### BACKGROUND

Workers in industries such as construction, manufacturing, mining, carpentry, auto repair, electrical work, plumbing, and welding face daily hazards that pose significant risks to their vision. Protective eyewear is a critical component of workplace safety, shielding workers from potential injuries caused by flying debris, chemical splashes, sparks, and excessive light exposure. Despite its importance, the process of selecting properly fitting protective eyewear remains outdated, often relying on manual measurements and subjective assessments by opticians. Ill-fitting eyewear can lead to discomfort, prescription misalignment, and reduced effectiveness in protecting against occupational hazards, increasing the likelihood of eye strain, fatigue, and workplace accidents. Given the stringent safety standards in these industries, ensuring precise, well-fitted eyewear is essential not only for compliance but also for enhancing worker safety, productivity, and overall well-being. As workplace environments evolve with technological advancements, the demand for more accurate, efficient, and AI-powered eyewear fitting solutions continues to grow, making innovation in this field both necessary and timely.

### SUMMARY

Traditional facial landmark detection methods often struggle to maintain real-time accuracy, particularly when users are blinking, moving, tilting their heads, or operating in suboptimal lighting conditions. These challenges can introduce errors that significantly impact the precision required for accurate eyewear fitting. To address this, the present disclosure employs a self-correction machine learning method (MLM) that continuously recalibrates facial landmark positions in real time. This advanced approach autonomously monitors for anomalies and dynamically adjusts without the need for manual user input. By correcting for landmark drift—minor positioning errors caused by movement or lighting variations—the present disclosure ensures sub-millimeter measurement accuracy, even in challenging conditions.

The present disclosure provides a system, method, and apparatus for a self-correcting, artificial intelligence (AI) powered facial landmark detection system designed for real-time, high-precision eyewear fitting and prescription lens alignment. Unlike conventional single-frame-based systems, this present disclosure utilizes multi-frame validation (operating at 60–240 FPS) to monitor and recalibrate facial landmark estimates dynamically. The system employs a combination of Kalman filtering for temporal smoothing, confidence-based landmark weighting, and machine learning-driven correction algorithms to detect and resolve landmark drift resulting from user motion, blinking, head tilt, or poor lighting. When anomalies are detected—such as a landmark shifting beyond a predefined threshold across frames—the system interpolates corrected positions using prior frame data, ensuring sub-millimeter measurement accuracy. Additionally, the present disclosure includes functionality for computing prescription-specific parameters such as segment height and ocular center height through Euclidean distance calculations based on dynamically adjusted landmarks. A hybrid architecture enables real-time performance on mobile devices via edge processing, with optional cloud-based refinement for enhanced precision.

In one exemplary aspect, a system is provided for a machine-learning based method for correcting facial landmark positions, the system including: an image sensor configured to perform a facial scan by capturing an image of a face of a user and generate a plurality of video frames from the captured image; and a processor coupled to the image sensor, the processor being configured to: extract facial landmark estimates corresponding to the face of the user from each of the plurality of video frames using a pre-trained facial landmark detection machine learning model (MLM) configured to identify anatomical features of the face of the user, wherein the extracted facial landmark estimates comprise at least one of: an eye estimate, a pupil estimate, or face width, store each of the extracted facial landmark estimates as a vector in a time-sequenced matrix, compare consecutive video frames from the plurality of video frames to detect facial landmark drift using a pre-trained self-correction MLM; recalibrate the extracted facial landmark estimates exhibiting drift using the pre-trained self-correction MLM; and recommend eyewear frames based on the extracted facial landmark estimates that do not exhibit drift and the recalibrated facial landmark estimates.

In some aspects, the techniques described herein relate to a system, wherein the pre-trained self-correction MLM is further configured to perform the recalibration on the extracted facial landmark estimates exhibiting drift by: applying a temporal smoothing function on a plurality of the extracted facial landmark estimates to recalibrate the extracted facial landmark estimates using a Kalman filter, generating confidence scores for the recalibrated facial landmark estimates based on at least one of: sharpness, consistency across video frames, clarity, symmetry and deviations from expected positions, and based on a determination that a confidence score for any one of the recalibrated facial landmark estimates falls below a predefined threshold, provide to the image sensor a rescan request.

In some aspects, the techniques described herein relate to a system, wherein processor is further configured to, based on a determination that drift persists more than a threshold number of consecutive frames, provide to the image sensor a rescan request.

In some aspects, the techniques described herein relate to a system, wherein the processor is further configured to provide to the image sensor a rescan request by sending to a display a message to re-adjust a facial angle of the user.

In some aspects, the techniques described herein relate to a system, wherein processor is further configured to, based on a determination that drift persists more than a threshold number of consecutive frames, apply to the image sensor a request to a brightness normalization.

In some aspects, the techniques described herein relate to a system, wherein processor is further configured to, based on a determination that a drift persists more than a threshold number of consecutive frames, re-align a position of the extracted facial landmark estimates with reference points.

In some aspects, the techniques described herein relate to a system, wherein processor is further configured to: generate an average segment height of the user based on the time-sequenced matrix and recommend prescription related measurements based on the average segment height.

In some aspects, the techniques described herein relate to a system, wherein the processor is further configured to: perform ocular center placement tracking based on the time-sequenced matrix and recommend prescription related measurements based on the ocular center placement.

In some aspects, the techniques described herein relate to a system, wherein the processor is further configured to: pre-train the facial landmark detection MLM using a facial landmark training dataset comprising at least one of: (a) facial images annotated with a distance between an outer or inner corners of both eyes, (b) facial images annotated with left and right boundaries of each iris, (c) facial images annotated with a pupil center for each eye, (d) facial images annotated with landmarks at a widest visible part of the face, (e) facial images annotated with a nasion point or a bottom of a nose bridge, and (f) ground truth labels for the facial images to serve as a target output for the facial landmark detection MLM.

In some aspects, the techniques described herein relate to a system, wherein the processor is further being configured to: pre-train the self-correction MLM using a self-correction training dataset comprising at least one of: (a) time-sequenced video frame data comprising a plurality of consecutive video frames, each frame labeled and annotated with ground truth facial landmark estimates to enable tracking of landmark positions across time, (b) temporally annotated datasets comprising facial landmark annotations with frame-to-frame smoothness indicators or velocity profiles to identify inconsistencies and abrupt deviations in facial landmark movement, (c) synthetically altered datasets comprising facial landmark annotations with artificial jitter, occlusion, or landmark drift introduced into otherwise smooth facial landmark sequences, paired with original ground truth landmarks as target outputs for self-correction training, (d) spatial and temporal landmark representations comprising structured encodings of facial geometry and frame-delta information to contextualize facial landmark changes across consecutive frames, (e) multimodal datasets comprising facial imagery, visual embeddings, head pose data, and synchronized audio or speech streams to support correlation between audio-visual cues and facial landmark dynamics, or (f) ground truth labels for the time-sequenced video frame data, temporally annotated datasets, synthetically altered datasets, spatial and temporal landmark representations, and multimodal datasets to serve as a target output for the self-correction MLM.

According to one aspect of the disclosure, a method for a machine-learning based method for correcting facial landmark positions is disclosed. The method includes: performing a facial scan by capturing an image of a face of a user and generating a plurality of video frames from the captured image, extracting a set of facial landmark estimates corresponding to the face of the user from each of the plurality of video frames using a pre-trained facial landmark detection machine learning model (MLM) configured to identify anatomical features of the face of the user, wherein the set of facial landmark estimates comprise at least eye measurements, pupil measurements, and face width, storing each of the facial landmark estimates as a vector in a time-sequenced matrix, validating the facial landmark estimates by comparing consecutive video frames from the plurality of video frames to detect facial landmark drift using a pre-trained self-correction MLM configured to track inconsistencies in detecting the facial landmark estimates using the vectors in the time-sequenced matrix, based on a determination that the pre-trained machine learning model detects facial landmark drift, generating recalibrated facial landmark estimates by performing recalibration on the consecutive frames using the pre-trained self-correction MLM and the time-sequenced matrix, and recommending eyewear frames based on the validated facial landmark estimates or the recalibrated facial landmark estimates.

The above simplified summary of example aspects serves to provide a basic understanding of the present disclosure. This summary is not an extensive overview of all contemplated aspects and is intended to neither identify key or critical elements of all aspects nor delineate the scope of any or all aspects of the present disclosure. Its sole purpose is to present one or more aspects in a simplified form as a prelude to the more detailed description of the disclosure that follows. To the accomplishment of the foregoing, the one or more aspects of the present disclosure include the features described and exemplarily pointed out in the claims.

### BRIEF DESCRIPTION OF THE DRAWINGS

The accompanying drawings, which are incorporated into and constitute a part of this specification, illustrate one or more example aspects of the present disclosure and, together with the detailed description, serve to explain their principles and implementations.

FIG. 1 is a block diagram illustrating a system for a measurement process, a calibration process, and an eyewear recommendation process according to aspects of the present disclosures.

FIG. 2 is a block diagram illustrating a system for continuously self-monitoring, detecting anomalies, and automatically recalibrating facial landmark estimates according to aspects of the present disclosure.

FIG. 3 is a block diagram illustrating a system preparing a machine learning model to extract a set of facial landmark estimates from a facial scan and to validate facial landmark estimates detected from the facial scan according to aspects of the present disclosure.

FIG. 4 is an example method for a machine-learning based method for correcting facial landmark positions according to an aspect of the present disclosure.

FIG. 5 presents an example of a general-purpose computer system on which aspects of the present disclosure can be implemented.

Like reference numbers and designations in the various drawings indicate like elements.

### DETAILED DESCRIPTION

Exemplary aspects are described herein in the context of a system, method, and computer program product for machine learning (ML)-based for performing continuous landmark validation and recalibration. Those of ordinary skill in the art will realize that the following description is illustrative only and is not intended to be in any way limiting. Other aspects will readily suggest themselves to those skilled in the art having the benefit of this disclosure. Reference will now be made in detail to implementations of the example aspects as illustrated in the accompanying drawings. The same reference indicators will be used to the extent possible throughout the drawings and the following description to refer to the same or like items.

Filling an eyeglass prescription involves a multi-step process that ensures users (e.g., patients) receive corrective lenses tailored to their specific vision needs. An eyeglass prescription is issued by an eyewear prescriber, such as an optometrist, following a comprehensive eye examination. This prescription contains the parameters required to fabricate lenses that correct visual impairments. Once the prescription is provided, the user is referred to an optician for frame selection, which is a crucial step in ensuring proper fit and comfort. Traditionally, opticians manually assess three key measurements: frame lens width, nose bridge width, and the overall length of the frame arm. However, rather than relying solely on precise measurements, many opticians conduct only a brief visual assessment and make frame recommendations based on personal judgment.

This traditional fitting process is often inefficient, time-consuming, and prone to inaccuracies, leading to frequent frame returns and dissatisfied customers. Poorly fitting eyewear can result in discomfort, misalignment of the prescription, and aesthetic concerns. Additionally, ill-fitting frames can pose safety hazards, particularly in workplaces where protective eyewear is necessary. Risks include eye strain, fatigue, reduced situational awareness, and persistent headaches, all of which can negatively impact productivity and well-being. In industries such as construction, manufacturing, and auto repair, improperly fitted eyewear fails to shield against light exposure, dust, and debris, increasing the likelihood of workplace accidents and associated compensation claims. These inefficiencies highlight the need for a more precise, data-driven approach to eyewear prescription fulfillment, ensuring a better fit, enhanced comfort, and improved safety for users.

By utilizing data-driven recommendations and ML models, the present disclosure eliminates guesswork, ensuring highly accurate frame selections tailored to each user’s face. This approach enhances efficiency, minimizes fitting errors, and significantly reduces high return rates associated with poorly fitting eyewear. In addition, the present disclosure improves the overall user experience, providing a seamless, accurate, and personalized eyewear fitting process.

The present disclosure utilizes an interactive video scan to capture 300-960 frames of a user’s face. In some aspects, the total number of frames depend on the user’s device capabilities. During the scanning process, the system collects 300-350 unique data points and facial measurements. These data points are processed using ML models which analyze the user’s face and determine the most precise frame fit. Unlike traditional approaches that rely on a fixed set of measurements, the system incorporates individualized algorithmic code to adapt dynamically to each user’s unique facial structure.

Most facial landmark systems analyze each frame independently, focusing solely on detecting static landmark positions in isolation. In contrast, the present disclosure is uniquely designed to track landmark motion over time, enabling it to observe temporal dynamics and trends. This temporal awareness allows the system to identify and correct inconsistencies that would otherwise go unnoticed (e.g., subtle drift or jitter) resulting in a more stable, accurate, and reliable performance across varying conditions.

Turning now to the figures, example aspects are depicted with reference to one or more components described herein, where components in dashed lines may be optional.

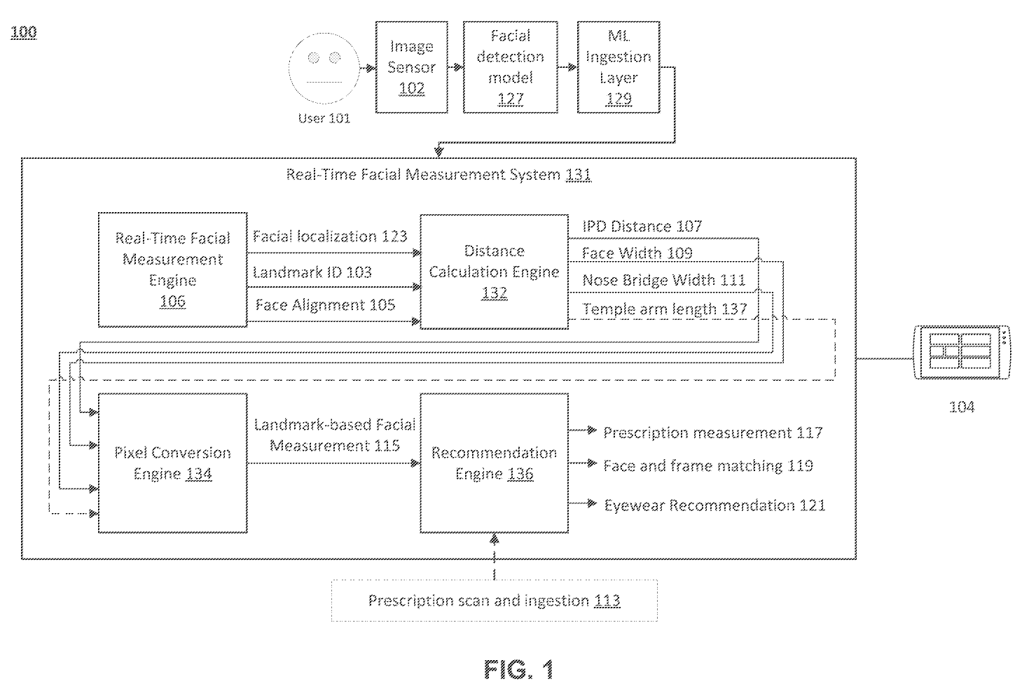

FIG. 1 is a block diagram illustrating a real-time facial measurement system 100 for implementing a measurement process and an eyewear recommendation process according to aspects of the present disclosure. In various implementations, the method of the system 100 is performed by a device with one or more processors and non-transitory memory that performs predictions. In some implementations, the method is performed by processing logic, including hardware, firmware, software, or a combination thereof.

An example of the system 100 may include an image sensor 102, a facial detection model 127, a ML ingestion layer 129, a real-time facial measurement system 131 including at least a real-time facial measurement engine 106, a distance calculation engine 132, a pixel conversion engine 134, and a recommendation engine 136, and a computing device 104 for displaying outputs. At a high level, the system 100 is configured to collect hundreds of facial data points during scanning, which are analyzed using generative artificial intelligence (AI) to determine the best-fitting eyewear frame(s) for a user. Unlike fixed-measurement methods, the system 131 dynamically adapts to each user’s unique facial structure. For example, the system 131 may convert pixel data into precise measurements, which are then matched against frame specifications. The system 131 further personalizes recommendations by factoring in profile data such as age, sex, ethnicity, and face shape. In some aspects, recommended frames are ranked by fit accuracy, and the platform continuously improves through self-learning, real-time data processing, and predictive modeling for future styles.

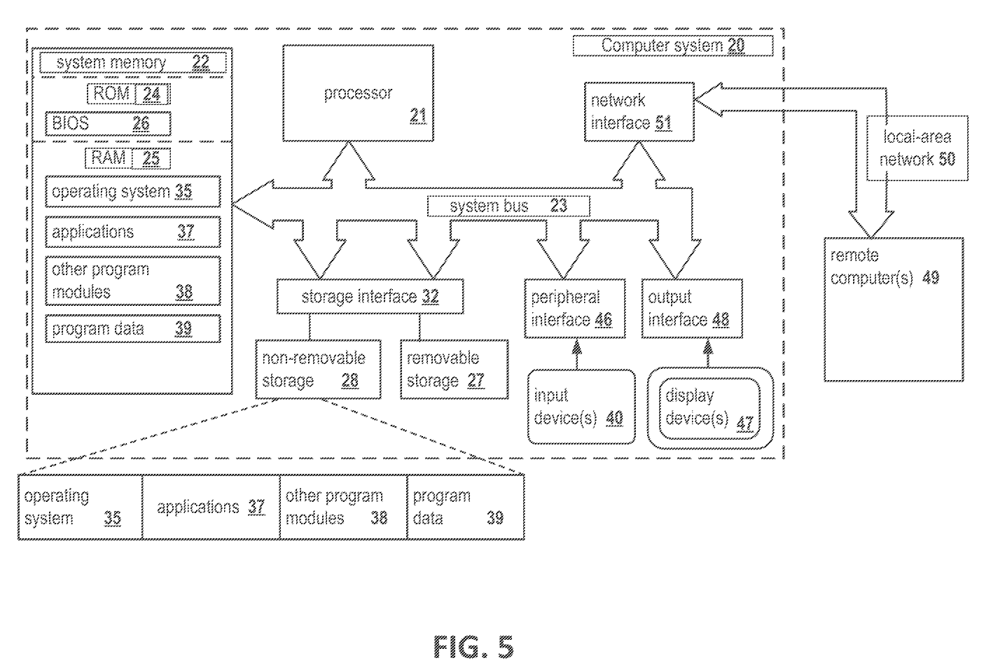

First, the image sensor 102 may be configured to perform a facial scan by capturing an image of a face of a user and generate a plurality of video frames from the captured image for performing continuous facial landmark tracking. A facial detection model 127 then processes each frame of the video data by performing face detection to identify a region of interest containing a user’s face. This region is then processed by the facial detection model 127 to output a set of hundreds of facial landmark estimates (e.g., 468 facial landmark estimates) for the user, each comprising normalized x, y, and z coordinates, where x and y represent 2D positions relative to the frame dimensions and z indicates relative depth. These facial landmark estimates correspond to measurements of anatomically significant facial points including the eyes, nose, lips, and facial contours (see FIG. 5). For each video frame, the system 131 outputs a complete set of landmark coordinates for the detected face(s). The process is repeated sequentially for all frames, enabling real-time or near-real-time tracking of facial features. Temporal inconsistencies, such as jitter or drift across frames, may occur due to variable lighting, head motion, or occlusions. Therefore, in some aspects, the facial landmark estimates output may be further refined using post-processing techniques such as temporal filtering, statistical smoothing, or machine learning-based correction to improve stability and reliability over time.

Specifically, the ML ingestion layer 129 functions as an intermediate processing stage that enhances landmark stability and anatomical accuracy. The ML ingestion layer 129 is configured to ingest raw landmark data (e.g., typically 468 facial points per frame) output from the facial detection model 127 and passes it through a supervised ML model (MLM) from the real-time facial measurement engine 106. In some aspects, the ML ingestion layer 129 is configured to perform at least one of: filtering out noise, correcting drift, and improving reliability of a set of facial landmark estimates comprising at least pupils, iris edge, a 68-point model, and temple-to-temple width. In some aspects, the ML ingestion layer 129 may generate a reliability score and/or an accuracy score across facial landmark sets. The output of the ML ingestion layer 129 is a refined and validated set of landmarks optimized for measurement precision. In addition, the ML ingestion layer 129 operates as a closed-loop system that continuously refines itself through feedback and ongoing data ingestion, enabling greater measurement accuracy, stability, and repeatability in vision-based facial analysis applications. The resulting data enables frame-accurate tracking of facial geometry, supporting further downstream applications such as measurement of interpupillary distance (IPD), iris diameter, and other pixel-based facial dimensions.

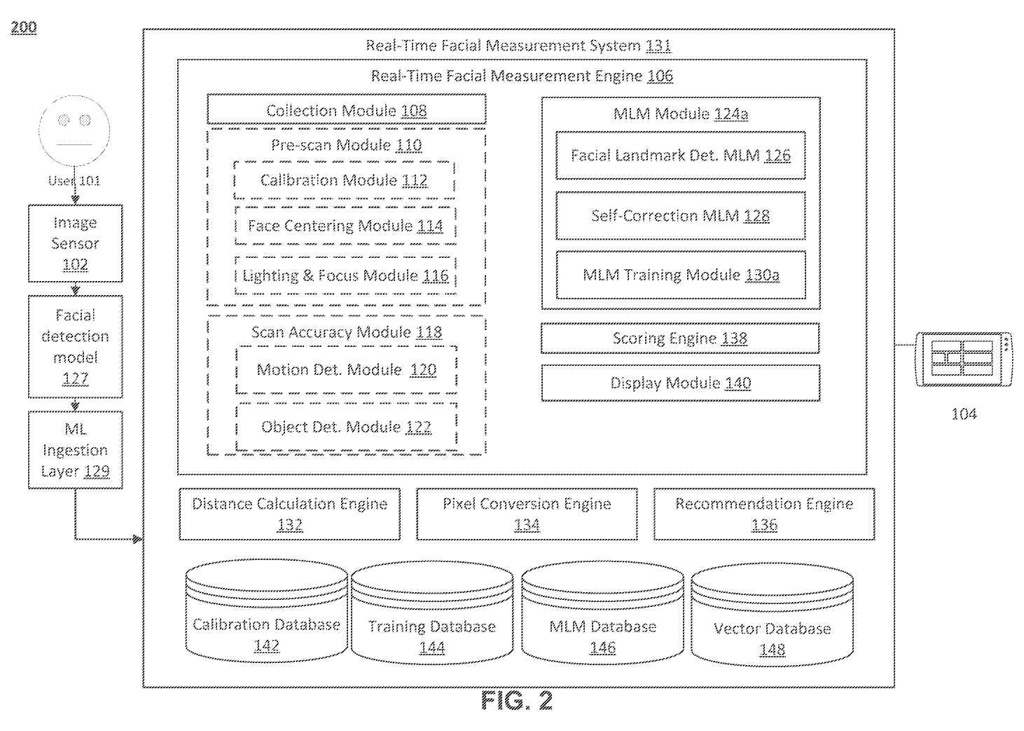

As will be described in more detail in FIG. 2 below, the real-time facial measurement system 131 includes at least a real-time facial measurement engine 106 configured to process video data of the user 101, facial landmark estimates extracted by the facial landmark/detection model 127 and/or refined dataset of the landmark output measurements from the facial detection model 127 to generate facial localization 123, landmark identification 103, and facial alignment 105. The facial localization process involves detecting the presence and position of a face of the user 101 within each frame of the video stream. For example, the facial localization process may involve identifying a bounding region that encapsulates the face using computer vision techniques or deep learning-based object detection models. Following localization, the real-time facial measurement engine 106 performs landmark identification 103 by detecting key facial features, such as the eyes, nose, mouth, and jawline, using a MLM configured to identify anatomical features of a face from frames of video data. These facial landmark estimates enable precise mapping of the user's facial geometry. Subsequently, facial alignment 105 is performed to normalize the orientation of the face. For example, this may include correcting for variations in head tilt, rotation, and scale. This alignment process uses the detected facial landmark estimates to geometrically transform the face to a standard pose, thereby enabling consistent downstream processing such as facial recognition, expression analysis, or biometric measurement.

The outputs generated by the real-time facial measurement engine 106 (e.g., including facial localization 123, landmark identification 103, and facial alignment 105) may be provided as inputs to a distance calculation engine 132 configured to compute precise facial dimensions such as interpupillary distance (IPD) 107, face width 109, nose bridge width 111, and optional temple arm length 137. In some embodiments, the landmark identification 103 process yields a set of spatial coordinates corresponding to predefined anatomical points on the face of the user 101, including the centers of the pupils, outer contours of the cheeks, and boundaries of the nasal bridge. For example, the system 131 may rely on a set of predefined facial landmark indices to accurately identify and track key anatomical regions of the face, which are essential for precise calculations in applications such as eyewear fitting. As will be shown in FIG. 5, critical facial points include the eye corners, iris points, temple to temple width markers, nose bridge points, temple arm length and pupil center points. Each of these indices corresponds to a specific region of the face, providing reliable reference points for determining spatial relationships and dimensions. Because the facial alignment 105 process standardizes the orientation and scale of the facial image data, these landmark coordinates can be reliably interpreted within a normalized reference frame. By leveraging these landmarks, the system 131 ensures accurate and personalized fitting of eyewear based on the user's unique facial structure.

In some aspects, the distance calculation engine 132 utilizes these normalized landmark positions to compute linear distances between specific pairs of points. For example, interpupillary distance 107 is calculated by determining the Euclidean distance between the landmarks corresponding to the centers of the left and right pupils. Similarly, temple to temple width 109 is computed as the distance between the outermost cheek landmarks, and nose bridge width 111 is determined by measuring the distance between the inner eye corners or nasal ridge landmarks. Optionally, a temple arm length 137 may also be calculated from landmarks by estimating a real-world distance along the side of the head from the front of the face near the eye to the back of the ear where the temple arms of glasses rest. These calculations may be performed in pixel space or, in some embodiments, converted to real-world units using a known reference scale or calibration technique by the pixel conversion engine 134. The resulting measurements may be used in downstream applications such as virtual eyewear fitting, biometric analysis, or customized product recommendations.

In some aspects, the interpupillary distance 107, temple to temple width 109, nose bridge width 111, and the optional temple arm length 137 may be provided as inputs to the pixel conversion engine 134 configured to generate landmark-based facial landmark estimates 115 in real-world or metric units (e.g., millimeters). These initial measurements, obtained in pixel space based on the spatial relationships between facial landmark estimates, are typically influenced by factors such as camera resolution, focal length, and user distance from the image sensor 102. The pixel conversion engine 134 may apply a transformation process that converts these pixel-based distances into calibrated metric units (e.g., millimeters or centimeters) by leveraging a reference scaling factor. This scaling factor may be determined through one or more calibration methods, such as using a known reference object within the image frame (e.g., a calibration marker or standardized object), leveraging device-specific camera metadata, or employing depth sensing technologies. Once the conversion factor is established, the pixel conversion engine 134 multiplies the raw pixel distances—such as the pixel-based interpupillary distance—by this factor to yield accurate, real-world measurements. The term “real-world measurements” refers to physical distances or dimensions expressed in standardized units that correspond to actual, physical quantities in the environment, including but not limited to metric units (e.g., millimeters, centimeters, meters, kilometers) and imperial units (e.g., inches, feet, yards, miles). Real-world measurements differ from pixel distances, which represent discrete counts of pixels within a digital image or display and lack intrinsic physical scale absent a defined conversion factor. The resulting landmark-based facial landmark estimates may include, but are not limited to, calibrated interpupillary distance 107, anatomical temple to temple width 109, nose bridge width 111, or optional temple arm length 137. These measurements can then be used in applications requiring precise sizing or fitting, such as augmented reality (AR) overlays, virtual try-on systems, biometric identification, or personalized wearable product recommendations.

In some embodiments, landmark-based facial estimates 115 and optional prescription scan and ingestion data are provided as inputs to a recommendation engine 136 configured to generate prescription-related measurement 117, perform face and frame matching 119, and output a personalized eyewear recommendation 121. The landmark-based facial estimates 115 may include, but are not limited to, IPD, temple to temple width, nose bridge width, ocular center height, and segment height, each derived from real-time facial landmark analysis. In some aspects, the optional prescription scan and ingestion module 113 may obtain prescription data from user-uploaded documents, electronic medical records, or images processed using optical character recognition (OCR). The optional prescription scan and ingestion module 113 is also configured to store both dynamic facial measurements and static frame measurements captured from selected video frames or high-resolution still images. These data inputs are used to generate advanced optical fitting parameters, including segment height (representing the vertical distance from the bottom edge of the virtual frame during scanning to the start of the near-vision zone in multifocal lenses) and ocular center height (indicating the vertical positioning of the pupil within the lens area).

The recommendation engine 136 uses this information to generate a prescription-related measurement 117 that ensures proper optical alignment based on both the user's prescription and their unique facial structure. Subsequently, the recommendation engine 136 conducts face and frame matching 119 by analyzing the compatibility of the facial measurements of the user 101 with a database of eyewear frames, evaluating parameters such as bridge width, lens height, frame width, and the vertical alignment of lenses with ocular centers. Based on these calculations and compatibility assessments, the recommendation engine 136 generates a personalized eyewear recommendation 121 optimized for the user's prescription accuracy, facial anatomy, comfort, and aesthetic fit. This recommendation may include specific frame models, lens configurations, and fitting adjustments, enabling a comprehensive and customized eyewear solution.

The applications of the real-time facial measurement system 131 may include at least ensuring bifocal and progressive lens are accurately positioned for vision correction, ensures eye tracking aligns properly with virtual reality headsets, ensures correct face symmetry analysis for reconstructive surgery, and tracks eye position in real-time to detect driver fatigue and distractions. By factoring in individual characteristics, the system 131 ensures a highly accurate and personalized eyewear recommendation. In some aspects, the system presents an eyewear selection ranked by percentage fit accuracy. Each frame is displayed hierarchically based on its form, fit, and function relative to the user’s unique facial structure. The system may continuously improve through: (1) self-learning AI algorithms that refine recommendations over time, (2) real-time data ingestion and processing, and (3) enhanced predictive modeling for future frame styles and fits. By leveraging generative AI, the system 131 evolves dynamically with every new scan to constantly improve its precision and adaptability.

The described system and methods provide several technical benefits that enhance the precision, efficiency, and personalization of eyewear fitting and prescription recommendation processes. For example, by leveraging real-time facial landmark detection, facial alignment, and pixel-to-metric conversion, the system enables accurate extraction of anatomical measurements such as interpupillary distance 107, temple to temple width 109, nose bridge width 111, optional temple arm length 137, segment height, and ocular center height, without requiring physical measurement tools or in-person fittings. The integration of prescription scan and ingestion functionality further streamlines the user experience by automating the capture and structuring of prescription data from various input sources, including scanned documents and electronic records. The combination of these data points within the recommendation engine 136 facilitates highly accurate prescription-related calculations, precise alignment of optical centers with user-specific eye positions, and optimal selection of eyewear frames based on individualized facial geometry. This results in improved visual clarity, comfort, and aesthetic appeal for the user 101. Additionally, the system reduces reliance on manual fitting methods, minimizes the potential for human error, and enables scalable, remote delivery of customized eyewear solutions, thereby improving accessibility and efficiency across tele-optometry and e-commerce eyewear platforms.

It should be noted that the various processes described in system 100 are described at a high level and further detail about the specific steps and processes will be described in further detail below.

FIG. 2 is a block diagram illustrating a system 200 for continuously self-monitoring, detecting anomalies, and automatically recalibrating facial landmark estimates according to aspects of the present disclosure. In one aspect, the components of system 200 may be implemented on computer systems, such as that shown in FIG. 5.

The present disclosure describes a real-time facial measurement system 131 including at least a real-time facial measurement engine 106 for detecting facial measurements using a deep learning-based MLM. Pre-trained on millions of facial images, the facial detection/landmark models 127 may detect 478+ facial landmark estimates in real time, covering key facial features such as the eyes, nose, mouth, and chin to enable comprehensive structural analysis. Unlike conventional systems that only capture 30 frames per second (FPS), the system 200 processes 60–240 FPS to continuously track micro-movements and reduce errors from motion blur or occlusions. Key frames are then selected using a weighted algorithm, and the coordinates of critical landmarks (e.g., eye corners, iris points, temple to temple width, nose bridge, and pupil center) are extracted for precise measurement. For example, using a smartphone camera (e.g., computing device 104 and image sensor(s) 102) operating at 120 FPS, the system 200 captures facial data, detects landmarks, and stores the (x, y) coordinates from the most accurate frames. To ensure sub-millimeter accuracy, the system 200 may optionally incorporate a calibration phase and real-time validation to verify optimal user position, lighting, focus, and stability. The scan pauses if conditions fall outside defined thresholds, preventing errors caused by poor positioning, excessive movement, blurriness, or foreign objects in the frame.

The system 100 includes at least a user 101, at least one image sensor 102 (e.g., as part of the computing device 104 or coupled to the computing device 104), a facial detection model 127, a ML ingestion layer 129, a real-time facial measurement system 131 including at least a real-time facial measurement engine 106, a distance calculating engine 132, a pixel conversion engine 134, and a recommendation engine 136, and a computing device 104. The facial detection model 127 may be configured to identify key facial regions and landmarks with high precision and low computational overhead by utilizing pre-trained machine learning model. In some aspects, the facial detection model 127 employs a pipeline architecture that includes face region proposal, landmark regression, and temporal smoothing to enable continuous and stable face tracking across frames. The ML ingestion layer 129 is configured to filter out noise, correct drift, and/or improve the reliability of the key facial regions and landmarks from the facial detection model 127. The real-time facial measurement engine 106 may be configured to generate prescription-related measurements for recommending eyewear based on video data capturing the face of the user 101. The computing device 104 may execute a plurality of modules in the real-time facial measurement engine 106 that together perform collection, calibration, analysis, self-correction, detection, calculation, conversion, and recommendation functions. In some aspects, the real-time facial measurement engine 106 may correspond to the computing device 104 or cloud network (not shown) that is configured to execute a plurality of modules that together make up the real-time facial measurement engine 106 for generating prescription-related measurements.

The real-time facial measurement engine 106 may be configured to validate facial landmark estimates or perform re-calibration on facial landmark estimates as part of an eyewear recommendation process. The computing device 104 may execute a plurality of modules in the real-time facial measurement engine 106 that together make up the collection, calibration, analysis, self-correction, detection, and recommendation system. In some aspects, the real-time facial measurement engine 106 may correspond to the computing device 104 or cloud network (not shown) that is configured to execute a plurality of modules that together make up the real-time facial measurement engine 106 for recommending eyewear based on validating facial landmark estimates or recalibrating facial landmark estimates.

In some aspects, the real-time facial measurement engine 106 may include at least a collection module 108, an optional pre-scan module 110 including at least an optional calibration module 112, an optional face centering module 114, and an optional lighting & focus module 116, an optional scan accuracy module 118 including an optional motion detection module 120 and an optional object detection module 122, a MLM module 124a including a facial landmark detection MLM 126, a self-correction MLM 128, and a MLM training module 130a, a recommendation engine 136, a scoring engine 138, and a display module 140. In some aspects, pre-scan measurements (e.g., user distance, face centering, lighting, and/or focus measurements) that ensure optimal scanning conditions may be stored on a calibration database 142. In some aspects, training data for the MLM training module 130a may be stored on a training database 144. In some aspects, a pre-trained facial landmark detection MLM 126 may be stored on a MLM database 146.In some aspects, the detected facial landmark estimates may be stored as a vector in a time-sequenced matrix in the vector database 148. In some aspects, the calibration database 142, training database 144, the MLM database 146, and/or the vector database 148 may be stored on a local device or a cloud network.

The computing device 104 may execute a collection module 108 to obtain a plurality of video frames via one or more image sensors 102 (e.g., from a smartphone or webcam) to continuously capture the face of a user 101 for high-precision facial landmark tracking. By utilizing advanced facial landmark processing, the system 200 performs high-frequency frame analysis at 60 to 240 frames per second (FPS), significantly exceeding the 30 FPS limit of older technologies and enabling finer detection of facial details. This elevated frame rate enables real-time, frame-by-frame landmark validation and correction, allowing the system 200 to accurately track even the smallest micro-movements of the face of the user 101. In this way, the system 200 may analyze multiple frames in sequence and selectively extract key frames with the clearest facial positioning to reduce the impact of motion blur and/or partial occlusions. This continuous, high-speed video capture ensures precise and reliable facial measurements, enhancing the overall effectiveness of the system 200.

The computing device 104 may execute an optional pre-scan module 110 to optimize scan conditions before the user begins recording their face for obtaining facial measurements. Specifically, the optional pre-scan module 110 may initiate a series of pre-scan calibration procedures designed to optimize environmental and user-related conditions prior to facilitate sub-millimeter precision in facial measurements. These preparatory measures ensure that the user 101 is correctly positioned at an appropriate distance and angle relative to the imaging sensor 102, while also verifying sufficient lighting and camera focus. The system 200 systematically evaluates multiple variables, including user proximity, facial alignment, ambient illumination levels, and image sharpness, to confirm that scanning conditions meet established quality benchmarks. This pre-scan validation process mitigates common sources of error such as improper user distance, blurriness, inadequate lighting, excessive head tilt, or visual obstructions within the frame. By enforcing these quality gates before initiating the scan, the system 200 significantly reduces the likelihood of distortion and ensures that only high-fidelity image data is captured for subsequent facial analysis and measurements.

Prior to initiating a facial scanning procedure, the system 200 may enter a calibration phase to verify that the user's position, distance, and facial orientation relative to the image-capturing device fall within predetermined optimal parameters. Specifically, an optional calibration module 112 may identify facial landmark estimates and determines a bounding box around the face of the user 101 to assess alignment within a defined spatial zone conducive to accurate biometric measurement. In addition, the optional calibration module 112 may also compute the real-world distance of the user 101 from the one or more image sensors 102 by estimating the interpupillary distance (IPD) in pixels and comparing it to an expected average IPD value (e.g., 64 mm). For example, the estimated distance may be computed as the known IPD x camera focal length divided by the detected IPD in pixels. In response to detecting a deviation from a predefined distance threshold (e.g., when the estimated user-to-camera distance is less than approximately 30 centimeters or exceeds approximately 80 centimeters), the system may activate a control routine configured to suspend the scanning operation. Concurrently, the system 100 may generate one or more user-facing prompts, which may be visual and/or auditory in nature, instructing the user 101 to adjust their position to fall within the acceptable distance range. Such calibration ensures that facial scans are conducted under conditions that minimize distortion and maximize reliability of biometric output.

The optional face centering module 114 may also implements a real-time facial bounding box tracking mechanism to continuously monitor the position of the user's 101 face relative to the center of the imaging frame. In some aspects, an artificial intelligence (AI) module such as the facial landmark detection MLM 126 may actively analyzes facial landmark data to ensure the face remains centrally aligned within a predefined bounding region. As an example, if more than approximately 30% of the facial area deviates beyond the bounds of this region, the system may initiate an automated interruption protocol that temporarily pauses the scanning process and presents the user with a prompt—visual, auditory, or both—instructing them to re-center their face. In parallel, a mathematical out-of-frame detection algorithm may compute the horizontal offset of the face center from the center of the display. As another example, if the calculated offset exceeds a threshold of approximately 30% of the total screen width, the system 100 flags an out-of-frame error and suspends the scan. As yet another example, on a display with a width of 1080 pixels, a detected face center at 900 pixels yields an offset of (900 - 540)/1080 = 33%, exceeding the threshold, and resulting in a scan pause and user guidance to reposition accordingly.

The optional lighting and focus module 116 may perform a pre-scan evaluation of lighting conditions and image focus clarity to optimize image fidelity and ensure the highest level of scan accuracy. As part of this process, the system 200 assesses the average luminance (L) within the detected facial region. If the measured brightness falls below a lower threshold (e.g., L < 50, indicating insufficient lighting) or exceeds an upper threshold (e.g., L > 220, indicating overexposure), the system 200 halts the scanning sequence and issues a prompt instructing the user 101 to adjust the ambient lighting conditions. For instance, a measured brightness level of 30 would trigger the system to pause and display a "Move to Better Lighting" notification. In addition to lighting assessment, the system 200 applies a blur detection algorithm based on Laplacian Variance analysis to evaluate image sharpness. If the resulting sharpness metric falls below a predetermined threshold (e.g., variance < 1000), indicative of a blurry image, the system interrupts the scan and generates a prompt advising the user to refocus the camera or stabilize their position. As an example, a calculated variance of 800 would activate this safeguard, ensuring that only well-illuminated and sharply focused images are processed for biometric analysis.

The computing device 104 may execute an optional scan accuracy module 118 to employ, during the facial scanning process, a real-time accuracy enforcement mechanism to continuously evaluate frame quality and ensure adherence to precision standards required for sub-millimeter facial measurement accuracy. The system 200 may actively monitor a range of dynamic conditions, including user position, facial clarity, stability, and the presence of foreign objects within the capture frame. If any frame fails to meet predefined quality thresholds (e.g., such as improper user distance, motion-induced blur, lighting inconsistencies, off-angle facial orientation, or obstructions affecting landmark detection), the system 200 temporarily halts the scan and prompts corrective user action before resuming. By filtering out suboptimal frames in real time, the system 200 ensures that only high-fidelity data is processed, thereby minimizing distortion and maximizing the integrity of biometric output.

Specifically, the optional motion detection module 120 may continuously monitor facial stability for the user. The optional motion detection module 120 may utilize the facial landmark detection MLM 126 to track key facial landmark estimates across sequential image frames to calculate displacement vectors to assess the degree and speed of user movement. For example, if the displacement of tracked points exceeds a predefined threshold (e.g., 2 millimeters between consecutive frame), the system 200 automatically pauses the scan to prevent data distortion. Additionally, the system 200 evaluates the velocity of motion (e.g., Motion Speed = (dframe2-dframe1)/∆t), and if the calculated movement speed exceeds approximately 3 millimeters per second, it flags the activity as excessive and prompts the user to remain still. For example, if the user shifts their head laterally by 4 millimeters within a time span of 0.1 seconds, the scan is interrupted, and the user is instructed to stay still. This enforcement mechanism ensures that only stable, high-quality frames are captured for biometric processing.

In addition, the optional object detection module 122 may identify and manage the presence of foreign objects (e.g., hands, glasses, or mobile phones) in the video frames to ensure accurate facial landmark detection. In some aspects, this process begins with object detection using the YOLOv5 model, which classifies all visible objects in the camera view. If non-facial objects are detected, the system 200 automatically pauses the scan to prevent interference. In addition, the system 200 includes occlusion handling. For example, if more than 20% of the face is obscured, the scan is halted, and the user is prompted to remove the obstruction. As another example, if glasses are detected during a scan not requiring them (e.g., a non-prescription scan), the system 200 will pause and notify the user to take them off before proceeding.

The computing device 104 may execute the MLM module 124a including at least a facial landmark detection MLM 126, a self-correction MLM 128, and a MLM training module 130a. The facial landmark detection MLM 126 is configured to identify anatomical features of a face for the user from each frame of the plurality of video frames. In addition, the facial landmark detection MLM 126 is also configured to select pairs of facial landmark estimates (e.g., center of left pupil and center of right pupil, a first outer iris edge and a second outer iris edge, or a first nose bridge point and a second nose bridge point). The self-correction MLM 128 is configured to continuously verify and adjust facial landmark positions in real-time.

There are several possible approaches that may be implemented using computer vision and machine learning models such as a neural network (e.g., a convolutional neural network (CNN) and/or a Graph Neural Network (GNN)) with a real-time, self-adjusting AI model. This hybrid approach enables the facial landmark detection MLM 126 to not only extract and interpret complex facial features with high accuracy but also adapt dynamically to changes in user behavior or environmental conditions, ensuring continuous precision throughout the scanning process.

A neural network is a type of machine learning process that uses interconnected nodes or neurons in a layered structure that resembles the human brain. The neural networks create an adaptive system that computers use to learn from their mistakes and improve continuously by comprehending unstructured data and make observations without explicit training. With neural networks, computers may distinguish and recognize images similar to humans. However, the neural networks in the facial landmark detection MLM 126 must first go through training to teach the neural networks to perform their respective specific tasks. It should be noted that CNN or GNN is described in this present disclosure for illustrative purposes only and that any suitable MLM may be utilized to perform the specific tasks of the Facial Landmark Detection MLM 128.

The machine learning module 130a may comprise one or more neural networks, which are a class of machine learning models inspired by the structure and functioning of the human brain. They consist of interconnected nodes, called neurons or artificial neurons, organized into layers. Neural networks are capable of learning complex patterns and representations from data. The neural network executed by MLM module 124a may be one of the following: transformer neural network, convolution neural network (CNN), Graph Neural Network (GNN), recurrent neural network (RNN), long short-term memory (LSTM) network, gated recurrent unit (GRU) network, autoencoder, generative adversarial network (GAN). CNNs are effective for image-related tasks because CNNs may automatically learn spatial hierarchies of features from the input images. For videos, RNNs or Long Short-Term Memory (LSTM) networks can be used to capture temporal dependencies between frames. In some aspects, a hybrid model may be used by combining CNNs for spatial feature extraction and RNNs for temporal analysis.

A CNN is specialized for processing grid-like data, such as images, and employs convolutional layers to learn spatial hierarchies of features, reducing the need for manual feature engineering. CNNs are well-suited for tasks like image classification, object detection, and image generation.

A GNN is a deep learning model that understands and learns from the connections between things, not just the things themselves. It’s ideal when the relationships between data points are just as important as the data points.

An autoencoder is a type of neural network used for unsupervised learning and dimensionality reduction and consists of an encoder that compresses input data into a lower-dimensional representation (encoding) and a decoder that reconstructs the original input from the encoding.

A GAN comprises a generator and a discriminator trained simultaneously through adversarial training. The generator aims to generate realistic data, while the discriminator tries to distinguish between real and generated data. A GAN is widely used for image and content generation tasks.

For computer vision tasks such as extracting a set of facial landmark estimates within video frames, an untrained facial landmark detection MLM 128 will first analyze the images from the training dataset (e.g., training database 144) to learn how to identify facial landmark estimates on the face of the user 101. As an example, the training dataset may include labeled dataset containing images or videos of faces of different people under different lighting conditions and varying color temperature.

During training of the facial landmark detection MLM 128, the training dataset will include at least facial images annotated with a distance between an outer or inner corners of both eyes of a user; facial images annotated with left and right boundaries of each iris; facial images annotated with a pupil center for each eye; facial images annotated with landmarks at the widest visible part of the face; and facial images annotated with a nasion point or a bottom of a nose bridge. The results from the untrained facial landmark detection MLM is then compared with known data set results using corresponding labels identifying at least a distance between an outer or inner corners of both eyes, left and right boundaries of each iris, a pupil center for each eye, the widest visible face of the user, or a nasion point or a bottom of a nose bridge. It should be noted that the input to the facial landmark detection MLM 128 will be the images from the training dataset 144.

For every input training sample from the training dataset, the neural network from the facial landmark detection MLM 128 will produce a prediction consisting of values representing the probability that a facial landmark detected is detected on an image of the face. The output with the highest probability determines the predicted pass or fail label. A class label for each input image is used to compute a loss (e.g., loss function).

The facial landmark detection MLM 128 then uses a loss function that quantifies the error between the predicted output and the ground truth for a given training sample. In other words, the loss function can be used to guide the learning process by updating the network weights in a way that improves the accuracy of future predictions. This process may continue until the difference between the prediction, and the correct targets is minimal. In some examples, an appropriate loss function, such as Mean Squared Error (MSE) for regression tasks (e.g., predicting brightness levels) or a Cross-Entropy Loss for classification tasks (e.g., detecting specific color changes).

In some aspects, an optimizer such as Adam or SGD may be used to train the models in the facial landmark detection MLM 128. In some aspects, the data may be split into training, validation, and test sets. In these aspects, the models from detection are trained on the training dataset and then validated by the validation sets in order to tune hyperparameters.

Once the neural network is trained (e.g., inference), the facial landmark detection MLM 128 may extract a set of facial landmark estimates from a frame containing the face of the user 101. Specifically, the facial landmark detection MLM 128 may contain a trained neural network configured to identify and detect facial landmark estimates in video frames capturing the face of the user.

During inference, the trained neural network model from facial landmark detection MLM 128 does not re-evaluate or adjust the layers of the neural network based on the results. Instead, the inference applies knowledge from the trained neural network and uses it to infer a result. Accordingly, when a new unknown dataset (e.g., video stream) is input through the trained neural network in the facial landmark detection MLM 128, the trained neural network outputs a prediction of whether a facial landmark is detected based on predictive accuracy of the neural network.

Similarly, to train a self-correction MLM 128 to track inconsistencies in facial landmark detection within a time-sequenced matrix, the training process begins with assembling a dataset composed of multiple sequences of consecutive video frames, each annotated with accurate ground truth facial landmark estimates. These sequences are used to capture the natural, temporally smooth movement of facial features across frames. To help the model learn to detect and correct errors, synthetic inconsistencies—such as random noise, misalignments, or dropped landmarks—are deliberately introduced into some of the annotated sequences, simulating the types of errors commonly encountered in real-world detection systems. The model is then trained to take as input a series of consecutive frames with potentially corrupted landmark data and to compare the landmark positions across time to identify anomalies that deviate from expected temporal patterns.

The input landmarks are encoded in a format that preserves spatial relationships and temporal context, such as sequences of coordinate vectors or graph-based representations. The model learns to validate the consistency of landmarks by examining the continuity and motion of facial key points over time—looking for unnatural jumps, missing points, or irregular motion. During training, it is supervised to reconstruct or "recalibrate" the landmarks, predicting the most likely correct positions based on both the temporal progression and spatial structure of the face. The training is guided by a loss function that penalizes deviation from ground truth landmarks and rewards smooth, plausible transitions between frames. As a result, the model develops the ability to autonomously detect and correct landmark inconsistencies by referencing adjacent frames, thereby producing recalibrated facial landmark estimates that maintain both spatial accuracy and temporal coherence in video-based facial analysis tasks.

For example, the self-correction training dataset may include at least one of: (a) time-sequenced video frame data comprising a plurality of consecutive video frames, each frame labeled and annotated with ground truth facial landmark estimates to enable tracking of landmark positions across time, (b) temporally annotated datasets comprising facial landmark annotations with frame-to-frame smoothness indicators or velocity profiles to identify inconsistencies and abrupt deviations in facial landmark movement, (c) synthetically altered datasets comprising facial landmark annotations with artificial jitter, occlusion, or landmark drift introduced into otherwise smooth facial landmark sequences, paired with original ground truth landmarks as target outputs for self-correction training, (d) spatial and temporal landmark representations comprising structured encodings of facial geometry and frame-delta information to contextualize facial landmark changes across consecutive frames, (e) multimodal datasets comprising facial imagery, visual embeddings, head pose data, and synchronized audio or speech streams to support correlation between audio-visual cues and facial landmark dynamics, or (f) ground truth labels for the time-sequenced video frame data, temporally annotated datasets, synthetically altered datasets, spatial and temporal landmark representations, and multimodal datasets to serve as a target output for the self-correction MLM 128.

The computing device 104 may execute a recommendation engine 136 to recommend eyewear based on the position of facial landmark estimates. For example, the recommendation engine 136 may analyze the detected facial landmark estimates (e.g., such as the positions of the eyes, nose bridge, cheekbones, temples, and jawline) captured from one or more images or video frames of the user 101. By evaluating the spatial relationships and dimensions derived from these landmarks, the recommendation engine 136 may determine the most suitable eyewear styles, sizes, and shapes that best fit the user's unique facial structure. The recommendation may further take into account proportions such as face width, eye distance, and face shape classification, enabling personalized suggestions that enhance comfort, aesthetics, and fit.

The computing device 104 may execute a scoring engine 138 to generate confidence scores for each detected facial landmark based on a combination of visual and spatial evaluation criteria. In some aspects, these criteria may include image sharpness, which reflects the clarity and focuses of the region surrounding a landmark; consistency across video frames, which measures how stable and coherent the landmark's position remains over time; and clarity, which assesses the visual distinctness of facial features in the image. Additionally, the scoring engine 138 may evaluate facial symmetry by comparing corresponding landmarks on opposite sides of the face to ensure balanced placement, and it may analyze deviations from expected anatomical positions to detect outliers or abnormal shifts. By synthesizing these indicators, the scoring engine 138 assigns a confidence score to each landmark, representing the system’s assessment of its accuracy and reliability for use in further processing, such as landmark refinement, tracking, or personalized applications like eyewear fitting.

The computing device 104 may execute a display module 140 to generate and display the recommended eyewear on the computing device 104. Generally, the display module 140 is responsible for managing and rendering the visual components of the user interface by handling the presentation of information to the user, ensuring that data and controls are displayed correctly and consistently across the UI.

In some aspects, the display module 140 is configured to render or draw all the elements of the UI, such as windows, buttons, text fields, menus, icons, images, and other components. In some aspects, the display module 140 is configured out update the UI when the data changes or user interactions occur (e.g., clicking a button or typing in a text box) such that the display module updates the UI accordingly. This could mean refreshing a portion of the screen, changing the state of a button, or displaying new data. In other words, the display module 140 may be considered the “view” part of a model-view-controller (MVC) or similar design pattern. It serves as the layer that presents data to the user and receives input to and from the computing device 104.

It should be noted that the collection, calibration, detection, self-correction, analysis, and recommendation modules described in the present disclosure are heavily simplified. One skilled in the art will appreciate that the MLMs utilized may have significantly large datasets with highly specific details far beyond the capabilities of the human mind and cannot be realistically performed using pen and paper due to the complexity, precision, and real-time processing requirements involved. These methods involve high-frequency processing of complex spatial and temporal data, such as analyzing up to 240 frames per second and comparing hundreds of facial landmark estimates per frame in real time. The level of precision required, often down to sub-millimeter accuracy, demands advanced mathematical modeling, including Kalman filtering, multi-frame validation, and machine learning-based anomaly detection—all of which exceed the capabilities of unaided human cognition. Additionally, recalibration relies on statistical interpolation and confidence scoring that synthesize visual sharpness, motion consistency, and symmetry across large datasets—operations that would be infeasible to perform manually due to the speed, scale, and precision involved. As such, the system's reliance on computing power and algorithmic decision-making makes it unsuitable for execution by a human or through manual methods.

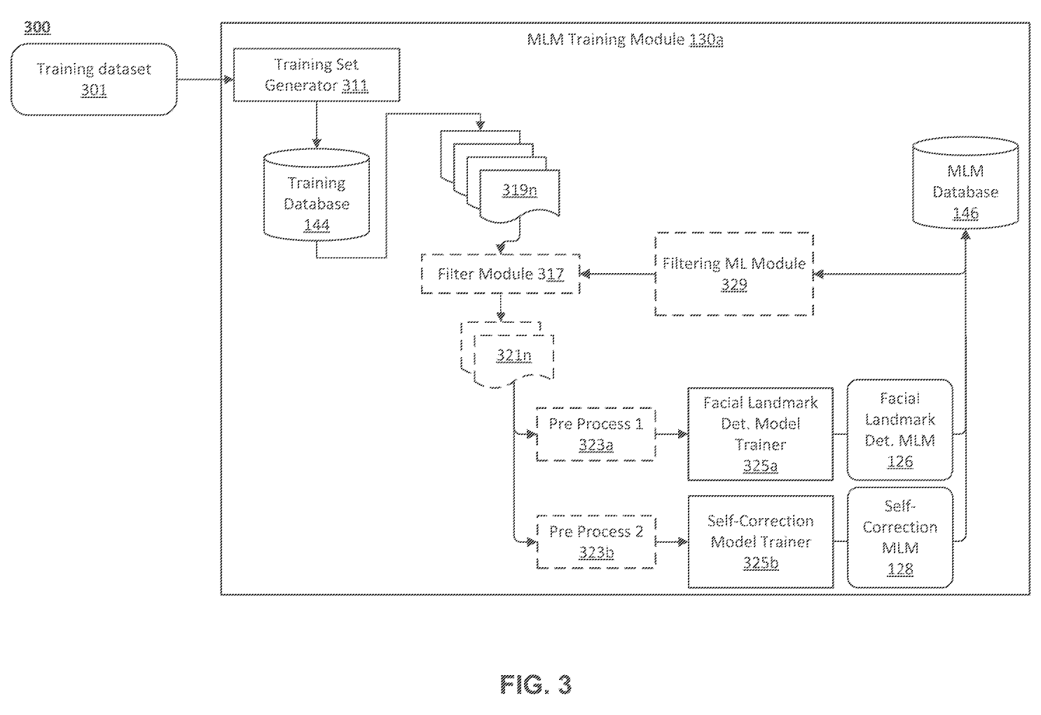

FIG. 3 is a block diagram illustrating a system preparing a machine learning model to extract a set of facial landmark estimates from a facial scan and to validate facial landmark estimates detected from the facial scan according to aspects of the present disclosure.

As shown in example 300, the MLM training module 130a is configured to build and train specialized machine learning models with inference to perform particular tasks. This enables the specialized machine learning models to develop an ability to perform particular objectives on inputs that are not part of a training dataset. By subjecting the specialized machine learning models to large amounts of unlabeled and/or labeled training data sets, the specialized machine learning models may perform particular tasks such as extracting a set of facial landmark estimates corresponding to the face of a user from each frame of the plurality of video frames, validating the facial landmark estimates by comparing consecutive video frames from the plurality of video frames to detect facial landmark drift, and/or generating recalibrated facial landmark estimates by performing recalibration on the consecutive frames.

Supervised learning is effective for tasks such as classification (assigning inputs to predefined categories) and regression (predicting continuous values) since it relies on the availability of labeled data for both training and evaluation phases. In supervised learning, the MLM training module 130a trains the algorithm on a labeled dataset, where each input has a corresponding output. The goal is to learn a mapping function from inputs to outputs, allowing the algorithm to make predictions or classifications on new, unseen data. The process typically involves the following steps: training, model building, prediction, feedback, and adjustment. In the training phase, the MLM training module 130a provides the algorithm with a training dataset including input-output pairs. The algorithm learns the mapping function that relates inputs to outputs through an iterative process, adjusting its internal parameters based on the provided examples.

During model building, the algorithm creates a model that can generalize from the training data to make predictions on new, unseen data. The model's complexity varies based on the algorithm used. For example, the model may be a simple linear regression model or a complex neural network. During the prediction phase, the MLM training module 130a inputs test inputs (i.e., inputs with known outputs) into the model, which generates predictions or classifications based on what it has learned during training. The accuracy of predictions is evaluated by comparing them to the known outputs in a validation or test dataset. During the feedback and adjustment phase, machine refines the model based on feedback from its predictions. If the predictions differ from the actual outputs, the algorithm adjusts its internal parameters to minimize the errors. The performance of the trained model is assessed using metrics such as accuracy, precision, recall, etc., depending on the nature of the problem.

In some aspects, the MLM training module 130a includes at least a training database 144 configured to store the raw training data 319n and corresponding labels, a MLM database 146 to store the trained models (e.g., the pre-trained facial landmark detection MLM 126 or the pre-trained self-correction MLM 128). In some aspects, the MLM training module 130a may include an optional filtering ML module 329 and an optional filter module 317 configured to filter data from the training database 144 for training by removing poorly generated training data.

Training data from the training dataset 301 is received into the MLM training module 130a via the training set generator 311. Details about the data included in each training dataset is described in more detail above with FIG. 1.

An optional filtering ML module 329 is configured to filter out bad training images and/or data in order to clean up the training data in the training dataset 319n. In some examples, the optional filter module 317 may be a neural network. In some examples, the optional filter module 317 is a mathematical model. In some examples, the cleaned training dataset 321n then undergoes optional preprocessing steps depending on which neural network or model is being trained.

The optional preprocess 1223a and optional preprocess steps 323b are automated processes that modifies the raw data received from 319n (or cleaned training dataset 321n) and prepare the raw data as input to the facial landmark detection model trainer 325a and the self-correction model trainer 325b, respectively. These may be described in the MLM training module 130a as snippets of code that prepares the datasets. In some examples, the preprocessing module (e.g., preprocess 323a or preprocess 323b) for a particular trainer may be an automated script or code that will be setup the first time any model is trained.

The facial landmark detection model trainer 325a and/or the self-correction model trainer 325b may be a script or code that train the facial landmark detection MLM 126 and the self-correction MLM 128. The facial landmark detection model trainer 325a and/or the self-correction model trainer 325b may be a script or code that holds the instructions on how a model should be trained (e.g., optimization method, model architecture, dataset division, etc.) and also runs the training. The facial landmark detection model trainer 325a and the self-correction model trainer 325b take as input the raw or filtered processed training data and train the facial landmark detection MLM 126 and self-correction MLM 128 to achieve their specific objectives, respectively.

In summary, the raw dataset 319n or cleaned dataset 321n may optionally go through preprocessing steps 323a and 323b and then a corresponding facial landmark detection model trainer 325a or self-correction model trainer 325b to generate a pre-trained facial landmark detection MLM 128 and pre-trained self-correction MLM 128. In some examples, each of these models may be a MLM or a neural network.

As a non-limiting example and as discussed above, the machine learning may be a neural network. The neural network models are designed using a set of hyperparameters that define high-level aspects of their architecture and training process. These hyperparameters include but are not limited to a combination of architecture type, number of layers, memory size, number of attention heads, learning rate, batch size, optimization algorithm, and the like. Based on these hyperparameters, learnable variables called parameters are initialized, which define the mathematical function that the neural network represents.

The raw training dataset 319n used for training may include noise and bad training images from the training database 144. Accordingly, to create a clean and filtered training dataset, the optional filter module 317 is configured to filter out unwanted data points from the raw training dataset 319n by developing smaller, less accurate systems based on patterns and metadata information.

During the training process, the facial landmark detection model trainer 325a and the self-correction model trainer 325b are presented with input data and labels of actual values, and the optimization objective, which aims to minimize the difference between the actual value and the predicted value, is calculated. The optimization algorithm updates the parameters of the facial landmark detection model trainer 325a and the self-correction model trainer 325b to reduce the value of the objective. This process is repeated for several iterations until the parameters do not change anymore. This process is repeated for various combinations of hyperparameters, and the model with the smallest label prediction error is selected as the final model.

When a new model (e.g., the pre-trained facial landmark detection MLM 126 and the pre-trained self-correction MLM 128) is created, and a new process for filtering and automated labeling is established, it is added to the MLM database 146 in the MLM training module 130a. This enables the new model to be part of the closed-loop model update process. Optionally, at regular intervals, data which is continuously collected can be filtered, labeled, and used to update old models by an optional filtering ML module 329. In some examples, the optional filtering ML module 329 is a neural network. In some examples, the optional filtering ML module 329 is a mathematical model. This approach may capture changes in the data over time.

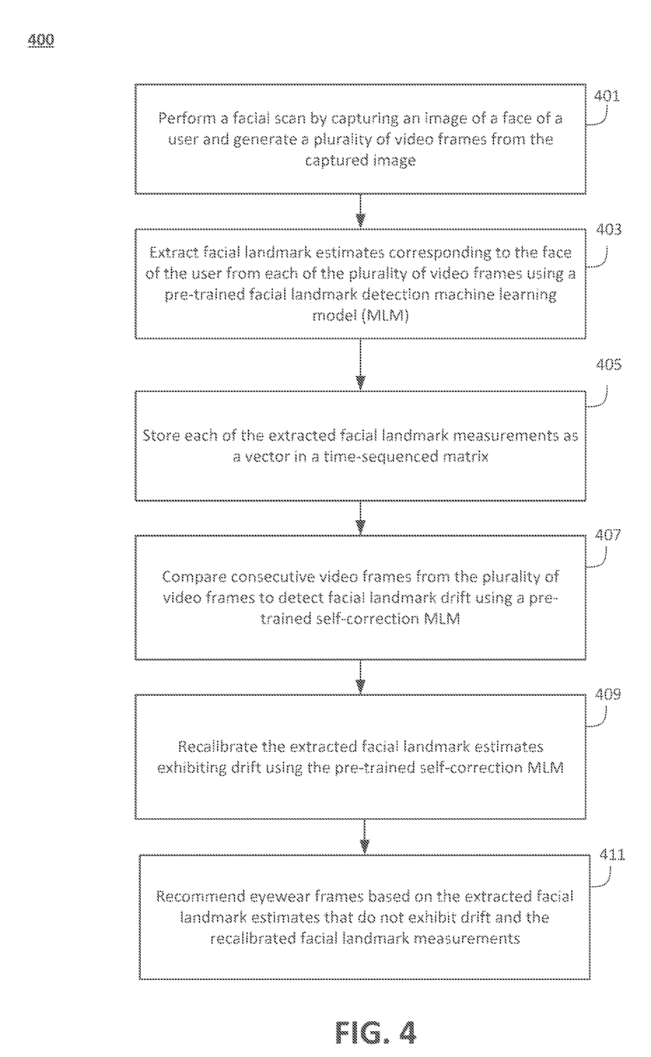

FIG. 4 is an example method for determining prescription-related facial measurements for fitting eyewear according to an aspect of the present disclosure. In various implementations, the method 400 is performed by a device with one or more processors and non-transitory memory that performs intent prediction. In some implementations, the method 400 is performed by processing logic, including hardware, firmware, software, or a combination thereof. In some implementations, the method 400 is performed by a processor executing code stored in a non-transitory computer-readable medium (e.g., a memory). The method 400 describes a method of for actively checking and recalibrating facial landmark positions in real time.

At 401, the method 400 may include performing a facial scan by capturing an image of a face of a user and generating a plurality of video frames from the captured image. This video feed provides the necessary input, allowing the system to identify and monitor key facial features in real time. The process serves as the foundation for subsequent analysis or interaction, ensuring that facial movements and expressions are consistently and accurately tracked throughout the session.

At 403, the method 400 may include extracting facial landmark estimates corresponding to the face of the user from each of the plurality of video frames using a pre-trained facial landmark detection machine learning model (MLM) configured to identify anatomical features of the face of the user. The extracted facial landmark estimates may include at least one of: an eye estimate, a pupil estimate, or face width. For example, the method 400 involves extracting facial landmark estimates from each frame of the captured video data using a pre-trained machine learning model. This model is specifically designed to recognize and identify anatomical features of the user's face with high accuracy. As the video is processed frame by frame, the system detects a consistent set of facial landmark estimates, which typically includes the eyes, pupils, and face width. These landmarks serve as critical reference points for further analysis, enabling applications such as facial recognition, alignment, and custom fitting tasks like eyewear measurement or augmented reality overlay.

At 405, the method 400 may include storing each of the facial landmark estimates as a vector in a time-sequenced matrix. The time-sequenced matrix is a fundamental component in accurately tracking how each landmark moves across a series of video frames. By organizing facial landmark positions over time, it provides the temporal context necessary to analyze motion in a meaningful way. For example, this structure is critical for detecting drift, which occurs when small errors in landmark detection accumulate over multiple frames, causing landmarks to slowly shift away from their true positions. It also plays a key role in smoothing jitter, or correcting for rapid, frame-to-frame fluctuations that result from noise rather than actual movement. These capabilities are essential for ensuring measurement stability, especially in applications where consistency and precision are paramount.

Without this matrix, each frame’s landmark data would be isolated, preventing us from assessing frame-to-frame consistency or applying temporal filters like Kalman filtering, which rely on historical data to make predictions and reduce noise. In such a case, landmark tracking would be significantly less accurate and far more prone to erratic behavior. The matrix structure enables our system to identify complex patterns, such as landmark drift occurring across three or more frames, and respond intelligently in real time. By leveraging this structure, the system can dynamically trigger smart behaviors—including automatic recalibration, temporal smoothing, or contextual user guidance—ensuring both accuracy and adaptability in rapidly changing scenarios.

Beyond simple tracking, the time-sequenced matrix transforms raw detection data into validated measurement outputs by embedding it within a timeline. This enables additional high-level functions such as drift detection, motion smoothing, and confidence scoring, which are essential for robust and stable tracking. Moreover, the matrix allows the system to recognize temporal patterns (i.e., identifying a gradual shift in a landmark’s position over three or more frames) and respond with intelligent, real-time actions. These might include triggering recalibration routines, apply adaptive smoothing, or provide guidance to the user if tracking reliability drops. In essence, the time-sequenced matrix is what makes dynamic, stable, and responsive landmark tracking possible.

At 407, the method 400 may include comparing consecutive video frames from the plurality of video frames to detect facial landmark drift using a pre-trained self-correction MLM.

In some aspects, the method 400 may include validating the facial landmark estimates by comparing consecutive video frames from the plurality of video frames to detect facial landmark drift using a pre-trained self-correction MLM configured to track inconsistencies in detecting facial landmark using the vectors in the time-sequenced matrix. The model operates on a time-sequenced matrix, which organizes landmark coordinates frame by frame, enabling temporal comparisons. By examining how each landmark shifts between consecutive frames, the MLM identifies patterns indicative of facial landmark drift—a gradual deviation from the expected position due to cumulative detection errors or instability in the tracking algorithm. When such drift is detected, the self-correction MLM can either flag the inconsistency for further processing or automatically adjust the landmark positions to restore alignment and maintain tracking accuracy. This validation step ensures greater measurement reliability by continuously monitoring for temporal inconsistencies and correcting them in real time.

At 409, the method 400 may include recalibrating the extracted facial landmark estimates exhibiting drift using the pre-trained self-correction MLM. Specifically, this step is triggered when the pre-trained self-correction MLM identifies inconsistencies in landmark positions across consecutive video frames, as tracked in the time-sequenced matrix. Once drift is detected, the model uses its learned understanding of typical facial movement patterns and temporal coherence to perform a recalibration process. This involves adjusting the landmark positions across the affected frames to realign them with expected trajectories, effectively correcting for the accumulated error. The recalibration leverages both the MLM’s predictive capabilities and the structured temporal data in the matrix to restore accuracy and continuity in the facial tracking. As a result, the system maintains stable and reliable landmark measurements, even in the presence of detection noise or model degradation over time.

In some aspects, the pre-trained self-correction MLM is hosted on a local device such as a smartphone or computer. This means the model operates entirely on the user's device rather than relying on cloud-based services. Hosting the model locally offers several important benefits. It enhances privacy by ensuring that user data remains on the device and is not transmitted to external servers. It also enables the system to function offline, providing consistent access to correction features without requiring an internet connection. Additionally, local deployment reduces latency, resulting in faster, real-time feedback for users. This approach can be more cost-effective by minimizing the need for cloud resources and also allows for greater personalization, as the model can adapt to individual user behavior without affecting other systems or users.

In some aspects, the pre-trained self-correction MLM is hosted on a cloud network. This setup allows the model to leverage the scalability, storage, and processing power of cloud infrastructure to deliver accurate and consistent text correction capabilities. By hosting the model in the cloud, updates and improvements can be deployed centrally, ensuring all users have access to the latest version without requiring manual updates on their devices. Additionally, the cloud-based approach supports seamless integration across multiple platforms and devices, enabling a more unified user experience. It also allows for more complex computations that might be too resource-intensive for local devices, making it suitable for handling large-scale or high-volume text processing tasks.

In some aspects, the pre-trained self-correction MLM is further configured to perform the recalibration on the extracted facial landmark estimates exhibiting drift by: applying a temporal smoothing function on a plurality of the extracted facial landmark estimates to recalibrate the extracted facial landmark estimates using a Kalman filter, generating confidence scores for the recalibrated facial landmark estimates based on at least one of: sharpness, consistency across video frames, clarity, symmetry and deviations from expected positions, and, based on a determination that a confidence score for any one of the recalibrated facial landmark estimates falls below a predefined threshold, provide to the image sensor a rescan request. For example, performing recalibration on consecutive frames using the pre-trained self-correction MLM and the time-sequenced matrix involves several key processes aimed at improving the accuracy and stability of facial landmark detection. This may include applying a temporal smoothing function, such as predicting the next probable position of each facial landmark using a Kalman filter, which helps reduce jitter and enhance motion continuity. Additionally, the system may generate confidence scores for each detected landmark based on factors such as image sharpness, consistency across video frames, clarity, facial symmetry, and deviations from expected anatomical positions. If the confidence score for a particular landmark fall below a predefined threshold, the system can respond by requesting additional frames to reassess the detection or, if necessary, halting further calculations to avoid inaccurate outputs. This approach helps maintain robustness and reliability in dynamic or uncertain visual environments.

In some aspects, the method 400 may further include, based on a determination that drift persists more than a threshold number of consecutive frames, provide to the image sensor a rescan request. In some aspects, the method 400 may include providing to the image sensor a rescan request by sending to a display a message to re-adjust a facial angle of the user. If it is determined that this drift—representing a gradual deviation from the expected or calibrated facial position—continues beyond a specified number of consecutive frames, the system may trigger an additional facial scan of the user. This additional scan serves as a recalibration step to realign the model’s understanding of the user's facial structure, ensuring accuracy and stability in ongoing analysis. By automatically initiating a re-scan when drift persists, the system helps maintain the reliability of performing facial measurements, particularly in dynamic conditions where small, accumulated inaccuracies could impact overall performance.

In some aspects, the method 400 may further include providing to the image sensor a rescan request by sending to a display a message to re-adjust a facial angle of the user. For example, the method 400 may further comprise transmitting, to the image sensor, a rescan request that is generated in response to detecting an inadequate or suboptimal facial pose during a prior image capture operation. The rescan request may include sending, to a display associated with the imaging device, a message or prompt instructing the user to re-adjust or modify a facial angle to improve alignment, orientation, or quality of the captured image data. Such a message may be configured to guide the user in repositioning their face relative to the image sensor, thereby enabling acquisition of facial images with improved pose suitability for subsequent processing, analysis, or authentication procedures.

In some aspects, the method 400 may further include based on a determination that drift persists more than a threshold number of consecutive frames, apply to the image sensor a request to a brightness normalization. Such a request may be configured to instruct the image sensor or associated processing circuitry to adjust illumination parameters, gain settings, or exposure values to compensate for lighting inconsistencies. By initiating brightness normalization in response to persistent drift, the method may enhance image quality and stability across frames, thereby facilitating improved accuracy in subsequent image analysis, recognition, or tracking operations.

In some aspects, the method 400 may further include, based on a determination that a drift persists more than a threshold number of consecutive frames, re-align a position of the extracted facial landmark estimates with reference points. For example, the method 400 may further include, upon determining that drift in the estimated facial landmark positions persists for more than a predetermined threshold number of consecutive frames, performing a re-alignment operation to correct the position of the extracted facial landmark estimates with respect to predefined reference points. This re-alignment may involve applying geometric transformations, recalibrating mapping functions, or adjusting coordinate offsets to reduce accumulated error and restore consistency between the detected landmarks and a reference facial model. Such an approach may improve the robustness and accuracy of facial landmark tracking across time, thereby enhancing performance in applications such as facial recognition, expression analysis, or augmented reality rendering.

At 411, the method 400 may include recommending eyewear frames based on the extracted facial landmark estimates that do not exhibit drift and the recalibrated facial landmark estimates. After the system has accurately identified and confirmed the positions of key facial features—such as the eyes, nose, and ears—through initial detection or subsequent recalibration, this precise facial data can be used to generate personalized eyewear recommendations. The validated or recalibrated landmarks ensure that the suggested eyewear fits the user's unique facial structure, enhancing both comfort and aesthetic appeal. By relying on accurate landmark positioning, the system can determine optimal frame dimensions, styles, and alignments that best suit the user’s face, providing a more tailored and satisfactory eyewear selection experience.

In some aspects, the method 400 may include generating an average segment height of the user based on the time-sequenced matrix and recommending prescription related measurements based on the average segment height. The prescription related measurements may be recommended based on the average seg height. For example, the average seg height may be calculated by analyzing temporal data reflecting the user's head or eye position over a period of time. Based on the determined average seg height, a recommendation for suitable eyewear can be made, ensuring optimal alignment and comfort tailored to the user's visual habits and posture.

In some aspects, the method 400 may include performing ocular center placement tracking based on the time-sequenced matrix and recommending prescription related measurements based on the ocular center placement. For example, this process may involve analyzing the user's eye position over a series of time-stamped data points to determine consistent patterns in ocular alignment. By identifying the average or most frequent placement of the ocular center during typical use, the system can recommend eyewear that aligns optimally with the user's natural eye positioning. Such alignment enhances visual clarity and comfort, ensuring that the eyewear supports the user's unique viewing behavior and ergonomic needs."

In some aspects, the method 400 may include generating a frame scoring for measuring the user based on the time-sequenced matrix. This process involves evaluating individual video frames within a sequence to determine their quality and reliability for accurate facial landmark detection. Each frame is assigned a score based on multiple factors, such as image clarity, lighting conditions, facial orientation, stability, and the visibility of key landmarks (e.g., pupils, nose bridge, and ears). Frames with higher scores are considered more suitable for precise measurement, while low-scoring frames—those that may be blurry, poorly lit, occluded, or captured during excessive head movement—are filtered out to avoid introducing noise or inaccuracies into the analysis. By applying this frame scoring mechanism, the method ensures that only the most reliable frames contribute to the measurement process, resulting in more accurate modeling of the user's facial features and more effective eyewear fitting or recommendation based on those measurements."

In some aspects, the method 400 may further include: pre-training the facial landmark detection MLM by: (1) providing, to facial landmark detection MLM, a facial landmark training dataset comprising at least one of: (a) facial images annotated with a distance between an outer or inner corners of both eyes of a user, (b) facial images annotated with left and right boundaries of each iris, (c) facial images annotated with a pupil center for each eye, (d) facial images annotated with landmarks at a widest visible part of the face, (e) facial images annotated with a nasion point or a bottom of a nose bridge, and (f) ground truth labels for the facial images to serve as a target output for the facial landmark detection MLM; and (2) preparing the facial landmark detection MLM using the provided facial landmark training dataset. This preparation involves two primary steps. First, the facial landmark detection MLM is provided with a dataset comprising facial images annotated with precise reference points critical for accurate landmark detection. These annotations may include: (a) distances between the outer or inner corners of the eyes, (b) the left and right boundaries of each iris, (c) the center point of each pupil, (d) landmarks along the widest visible part of the face, (e) the nasion point or the bottom of the nose bridge, and (f) ground truth labels corresponding to these landmarks to serve as target outputs. Second, the facial landmark detection MLM is trained or fine-tuned using this annotated dataset, enabling it to learn the spatial relationships and patterns necessary for high-precision facial landmark identification. This preparation process ensures that the model is well-equipped to detect key facial features accurately in real-world conditions, supporting downstream tasks such as frame fitting, ocular center tracking, and segment height measurement.

In some aspects, the method 400 may further include: pre-training the self-correction MLM by: (1) providing, to the self-correction MLM, a self-correction training dataset comprising at least one of: (a) time-sequenced video frame data comprising a plurality of consecutive video frames, each frame labeled and annotated with ground truth facial landmark estimates to enable tracking of landmark positions across time, (b) temporally annotated datasets comprising facial landmark annotations with frame-to-frame smoothness indicators or velocity profiles to identify inconsistencies and abrupt deviations in facial landmark movement, (c) synthetically altered datasets comprising facial landmark annotations with artificial jitter, occlusion, or landmark drift introduced into otherwise smooth facial landmark sequences, paired with original ground truth landmarks as target outputs for self-correction training, (d) spatial and temporal landmark representations comprising structured encodings of facial geometry and frame-delta information to contextualize facial landmark changes across consecutive frames, (e) multimodal datasets comprising facial imagery, visual embeddings, head pose data, and synchronized audio or speech streams to support correlation between audio-visual cues and facial landmark dynamics, or (f) ground truth labels for the time-sequenced video frame data, temporally annotated datasets, synthetically altered datasets, spatial and temporal landmark representations, and multimodal datasets to serve as a target output for the self-correction MLM; and (2) preparing the self-correction MLM using the provided self-correction training dataset. This preparation process involves two key steps. First, the self-correction MLM is trained using a comprehensive self-correction training dataset that may include: (a) time-sequenced video frame data with each frame annotated with ground truth facial landmark estimates to enable consistent landmark tracking across time; (b) temporally annotated datasets containing facial landmark sequences with indicators of smoothness or velocity to highlight irregular or abrupt changes in landmark motion; (c) synthetically altered datasets where artificial jitter, occlusion, or drift are introduced into facial landmark sequences, paired with the original ground truth annotations to teach the model to recognize and correct distortions; (d) structured spatial and temporal landmark representations that encode geometric relationships and frame-to-frame changes, helping the model learn contextual patterns of natural facial movement; (e) multimodal datasets combining visual data with additional inputs such as head pose, visual embeddings, or synchronized audio, enabling the model to use cross-modal correlations for improved prediction accuracy; and (f) ground truth labels across all these data types, serving as the target outputs for model training. Second, the self-correction MLM is trained using this diverse and richly annotated dataset, enabling it to detect inconsistencies in facial landmark tracking—such as sudden jumps or drifts—and autonomously correct them, resulting in smoother, more reliable facial landmark trajectories across video sequences. This capability is crucial for applications requiring high temporal consistency in facial measurements, such as eyewear fitting, user identification, or gesture recognition.

It is understood that the method illustrated by FIG. 4 is exemplary in nature and that the steps described herein may be combined or modified to generate alternative embodiments.

FIG. 5 is a block diagram illustrating a computer system 20 on which aspects of systems and methods machine learning (ML)-based analysis of determining prescription-related facial measurements for fitting eyewear may be implemented. The computer system 20 can be in the form of multiple computing devices, or in the form of a single computing device, for example, a desktop computer, a notebook computer, a laptop computer, a mobile computing device, a smart phone, a tablet computer, a server, a mainframe, an embedded device, and other forms of computing devices.

As shown, the computer system 20 includes a central processing unit (CPU) 21, a system memory 22, and a system bus 23 connecting the various system components, including the memory associated with the central processing unit 21. The system bus 23 may comprise a bus memory or bus memory controller, a peripheral bus, and a local bus that is able to interact with any other bus architecture. Examples of the buses may include PCI, ISA, PCI-Express, HyperTransport™, InfiniBand™, Serial ATA, I2C, and other suitable interconnects. The central processing unit 21 (also referred to as a processor) can include a single or multiple sets of processors having single or multiple cores. The processor 21 may execute one or more computer-executable code implementing the techniques of the present disclosure. For example, any of commands/steps discussed in FIGS. 1-6 may be performed by processor 21. The system memory 22 may be any memory for storing data used herein and/or computer programs that are executable by the processor 21. The system memory 22 may include volatile memory such as a random access memory (RAM) 25 and non-volatile memory such as a read only memory (ROM) 24, flash memory, etc., or any combination thereof. The basic input/output system (BIOS) 26 may store the basic procedures for transfer of information between elements of the computer system 20, such as those at the time of loading the operating system with the use of the ROM 24.

The computer system 20 may include one or more storage devices such as one or more removable storage devices 27, one or more non-removable storage devices 28, or a combination thereof. The one or more removable storage devices 27 and non-removable storage devices 28 are connected to the system bus 23 via a storage interface 32. In an aspect, the storage devices and the corresponding computer-readable storage media are power-independent modules for the storage of computer instructions, data structures, program modules, and other data of the computer system 20. The system memory 22, removable storage devices 27, and non-removable storage devices 28 may use a variety of computer-readable storage media. Examples of computer-readable storage media include machine memory such as cache, SRAM, DRAM, zero capacitor RAM, twin transistor RAM, eDRAM, EDO RAM, DDR RAM, EEPROM, NRAM, RRAM, SONOS, PRAM; flash memory or other memory technology such as in solid state drives (SSDs) or flash drives; magnetic cassettes, magnetic tape, and magnetic disk storage such as in hard disk drives or floppy disks; optical storage such as in compact disks (CD-ROM) or digital versatile disks (DVDs); and any other medium which may be used to store the desired data and which can be accessed by the computer system 20.

The system memory 22, removable storage devices 27, and non-removable storage devices 28 of the computer system 20 may be used to store an operating system 35, additional program applications 37, other program modules 38, and program data 39. The computer system 20 may include a peripheral interface 46 for communicating data from input devices 40, such as a keyboard, mouse, stylus, game controller, voice input device, touch input device, or other peripheral devices, such as a printer or scanner via one or more I/O ports, such as a serial port, a parallel port, a universal serial bus (USB), or other peripheral interface. A display device 47 such as one or more monitors, projectors, or integrated display, may also be connected to the system bus 23 across an output interface 48, such as a video adapter. In addition to the display devices 47, the computer system 20 may be equipped with other peripheral output devices (not shown), such as loudspeakers and other audiovisual devices.

The computer system 20 may operate in a network environment, using a network connection to one or more remote computers 49. The remote computer (or computers) 49 may be local computer workstations or servers comprising most or all of the aforementioned elements in describing the nature of a computer system 20. Other devices may also be present in the computer network, such as, but not limited to, routers, network stations, peer devices or other network nodes. The computer system 20 may include one or more network interfaces 51 or network adapters for communicating with the remote computers 49 via one or more networks such as a local-area computer network (LAN) 50, a wide-area computer network (WAN), an intranet, and the Internet. Examples of the network interface 51 may include an Ethernet interface, a Frame Relay interface, SONET interface, and wireless interfaces.

Aspects of the present disclosure may be a system, a method, and/or a computer program product. The computer program product may include a computer readable storage medium (or media) having computer readable program instructions thereon for causing a processor to carry out aspects of the present disclosure.

As used herein, the term “processor” is intended to be interpreted broadly and may refer to any hardware, firmware, software, or combination thereof configured to carry out one or more of the described operations, including, without limitation, signal processing, artificial intelligence functions, machine learning, deep learning, neural network inference, training, feature extraction, pattern recognition, and recommendation generation. A processor may be implemented using, without limitation, a general-purpose processor (GPP) such as a microprocessor or central processing unit (CPU), a digital signal processor (DSP), an application-specific integrated circuit (ASIC), a field-programmable gate array (FPGA) or other programmable logic device (PLD), a neural network accelerator, tensor processing unit (TPU), or other AI-specific processing unit, a graphics processing unit (GPU) configured for parallel or matrix computations, a microcontroller, a state machine, discrete gate or transistor-level logic, or any combination thereof, including configurations in which functions are split or shared among multiple components. The processor may be embodied as a single computing device or distributed across multiple devices or entities operating together. For example, processing may be performed by a combination of one or more microprocessors, one or more DSPs, and one or more neural network engines operating cooperatively or in parallel. In certain embodiments, the processor may be implemented in a heterogeneous system-on-chip (SoC) including CPU, DSP, GPU, and AI cores. The term "processor" should be understood to encompass multi-core, multi-threaded, or virtual processing environments, and may operate in conjunction with or under the control of software and/or firmware instructions stored in a non-transitory computer-readable medium. Unless expressly limited, no specific architecture, instruction set, or processing model is required. All such configurations, whether now known or later developed, that are capable of performing the described functionality are within the scope of the term "processor" as used herein.

A non-transitory computer-readable medium may store instructions for execution by one or more processors to perform any of the methods, processes, algorithms, or functions described herein. The non-transitory computer-readable medium described herein may be located on, off, or distributed with respect to the processor or processors that execute the instructions stored thereon. For example, the medium may include on-chip memory components, such as registers, level 1 (L1) or level 2 (L2) caches, or other memory blocks physically integrated with the processor (e.g., in a system-on-chip (SoC) or multi-core architecture). Alternatively or additionally, the medium may include off-chip or external memory components, such as random access memory (RAM), read-only memory (ROM), flash memory, solid-state drives (SSDs), hard drives, or networked or remote storage systems, including cloud-based or distributed storage architectures. The non-transitory computer-readable medium may also be logically or physically distributed across multiple memory devices or components. For example, a portion of the instructions may be stored in cache on the processor, while other portions reside in external memory modules or remote memory systems accessible via a bus or network interface. In some embodiments, the medium may include storage that is shared across multiple processors (e.g., shared memory in a multi-core environment), or distributed between processors and peripheral components, such as co-processors, accelerators, or I/O controllers. The computer-readable medium may further store data sets, machine learning models, parameters, or other information used in connection with artificial intelligence-based image processing, facial analysis, or recommendation generation. Accordingly, the computer-readable medium may be part of the processor, reside external to the processor, or be distributed across both, including within or across one or more computing entities, such as a combination of CPUs, DSPs, GPUs, neural network accelerators, and associated memory hierarchies as previously defined herein. The architecture may be implemented in a centralized, decentralized, or distributed computing environment, without limitation, and the term "non-transitory computer-readable medium" should be interpreted broadly to encompass all such configurations.

The computer readable storage medium can be a tangible device that can retain and store program code in the form of instructions or data structures that can be accessed by a processor of a computing device, such as the computing system 20. The computer readable storage medium may be an electronic storage device, a magnetic storage device, an optical storage device, an electromagnetic storage device, a semiconductor storage device, or any suitable combination thereof. By way of example, such computer-readable storage medium can comprise a random access memory (RAM), a read-only memory (ROM), EEPROM, a portable compact disc read-only memory (CD-ROM), a digital versatile disk (DVD), flash memory, a hard disk, a portable computer diskette, a memory stick, a floppy disk, or even a mechanically encoded device such as punch-cards or raised structures in a groove having instructions recorded thereon. As used herein, a computer readable storage medium is not to be construed as being transitory signals per se, such as radio waves or other freely propagating electromagnetic waves, electromagnetic waves propagating through a waveguide or transmission media, or electrical signals transmitted through a wire.

Computer readable program instructions described herein can be downloaded to respective computing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network interface in each computing device receives computer readable program instructions from the network and forwards the computer readable program instructions for storage in a computer readable storage medium within the respective computing device.

Computer readable program instructions for carrying out operations of the present disclosure may be assembly instructions, instruction-set-architecture (ISA) instructions, machine instructions, machine dependent instructions, microcode, firmware instructions, state-setting data, or either source code or object code written in any combination of one or more programming languages, including an object-oriented programming language, and conventional procedural programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a LAN or WAN, or the connection may be made to an external computer (for example, through the Internet). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

In various aspects, the systems and methods described in the present disclosure can be addressed in terms of modules. The term "module" as used herein refers to a real-world device, component, or arrangement of components implemented using hardware, such as by an application specific integrated circuit (ASIC) or FPGA, for example, or as a combination of hardware and software, such as by a microprocessor system and a set of instructions to implement the module’s functionality, which (while being executed) transform the microprocessor system into a special-purpose device. A module may also be implemented as a combination of the two, with certain functions facilitated by hardware alone, and other functions facilitated by a combination of hardware and software. In certain implementations, at least a portion, and in some cases, all, of a module may be executed on the processor of a computer system. Accordingly, each module may be realized in a variety of suitable configurations and should not be limited to any particular implementation exemplified herein.

As used herein, the term “image sensor” refers to any device, circuit, system, or component that is capable of capturing, detecting, acquiring, or converting an optical image, light signal, or visual representation into an electrical signal or digital data. The image sensor may be implemented using charge-coupled device (CCD) technology, complementary metal-oxide-semiconductor (CMOS) technology, active pixel sensors (APS), photodiode arrays, avalanche photodiodes, quantum dot-based sensors, organic photodetectors, infrared (IR) sensors, ultraviolet (UV) sensors, time-of-flight (ToF) sensors, lidar-based sensors, thermal imaging sensors, or any other technology now known or later developed that is capable of capturing an image or light information. The term "image sensor" also includes, without limitation, multi-modal sensors capable of capturing data across multiple spectral bands (e.g., visible, infrared, ultraviolet), depth sensors, 3D imaging sensors, structured light sensors, stereo cameras, and any sensor capable of acquiring biometric or anthropometric data relevant to facial analysis. In some embodiments, the image sensor may include or be associated with optical elements such as lenses, shutters, filters, or microlens arrays; signal conditioning components such as analog-to-digital converters or amplifiers; and control logic, which may be implemented via firmware, software, or hardware, to facilitate acquisition and processing of image data. The image sensor may operate under the control of a processor (as defined herein) and may be locally or remotely situated relative to such processor. The image sensor may be implemented as a single unit, a distributed array, or a plurality of cooperating sensors.

As used herein, the term “display” refers to any device, component, system, or interface configured to present visual information to a user or viewer, including but not limited to images, graphics, text, video, icons, user interface elements, or any other visually perceptible data. The display may include, without limitation, a liquid crystal display (LCD), light-emitting diode (LED) display, organic light-emitting diode (OLED) display, quantum-dot display (QLED or QD-OLED), plasma display, electrophoretic display (e.g., e-ink), microLED display, projection-based display, laser-based display, holographic display, head-up display (HUD), retinal projection display, 3D display, volumetric display, augmented reality (AR) or virtual reality (VR) head-mounted display, or any other display technology now known or later developed. The term "display" further includes any device or system capable of presenting augmented reality (AR) or virtual reality (VR) content, including overlays of virtual eyeglass frames on a user's face, or otherwise facilitating interactive visualization of recommended products. As used herein, the term "display" encompasses single-screen displays, multi-screen displays, curved or flexible displays, transparent displays, and modular display systems, as well as any associated control electronics, display drivers, timing controllers, graphics processing units (GPUs), backlight units, or optical components necessary to support image rendering and visibility.

In the interest of clarity, not all of the routine features of the aspects are disclosed herein. It would be appreciated that in the development of any actual implementation of the present disclosure, numerous implementation-specific decisions must be made in order to achieve the developer’s specific goals, and these specific goals will vary for different implementations and different developers. It is understood that such a development effort might be complex and time-consuming but would nevertheless be a routine undertaking of engineering for those of ordinary skill in the art, having the benefit of this disclosure.

Furthermore, it is to be understood that the phraseology or terminology used herein is for the purpose of description and not of restriction, such that the terminology or phraseology of the present specification is to be interpreted by the skilled in the art in light of the teachings and guidance presented herein, in combination with the knowledge of those skilled in the relevant art(s). Moreover, it is not intended for any term in the specification or claims to be ascribed an uncommon or special meaning unless explicitly set forth as such.

The various aspects disclosed herein encompass present and future known equivalents to the known modules referred to herein by way of illustration. Moreover, while aspects and applications have been shown and described, it would be apparent to those skilled in the art having the benefit of this disclosure that many more modifications than mentioned above are possible without departing from the inventive concepts disclosed herein.

What is claimed is:

1. A system for correcting facial landmark positions, comprising:
an image sensor configured to perform a facial scan by capturing an image of a face of a user and generate a plurality of video frames from the captured image; and
a processor coupled to the image sensor, the processor being configured to:
extract facial landmark estimates corresponding to the face of the user from each of the plurality of video frames using a pre-trained facial landmark detection machine learning model (MLM) configured to identify anatomical features of the face of the user, wherein the extracted facial landmark estimates comprise at least one of: an eye estimate, a pupil estimate, or face width,
store each of the extracted facial landmark estimates as a vector in a time-sequenced matrix,
compare consecutive video frames from the plurality of video frames to detect facial landmark drift using a pre-trained self-correction MLM,
recalibrate the extracted facial landmark estimates exhibiting drift using the pre-trained self-correction MLM, and
recommend eyewear frames based on the extracted facial landmark estimates that do not exhibit drift and the recalibrated facial landmark estimates.

2. The system of claim 1, wherein the pre-trained self-correction MLM is further configured to perform the recalibration on the extracted facial landmark estimates exhibiting drift by:
applying a temporal smoothing function on a plurality of the extracted facial landmark estimates to recalibrate the extracted facial landmark estimates using a Kalman filter,
generating confidence scores for the recalibrated facial landmark estimates based on at least one of: sharpness, consistency across video frames, clarity, symmetry and deviations from expected positions, and
based on a determination that a confidence score for any one of the recalibrated facial landmark estimates falls below a predefined threshold, provide to the image sensor a rescan request.

3. The system of claim 1, wherein processor is further configured to, based on a determination that drift persists more than a threshold number of consecutive frames, provide to the image sensor a rescan request.

4. The system of claim 3, wherein the processor is further configured to provide to the image sensor a rescan request by sending to a display a message to re-adjust a facial angle of the user.

5. The system of claim 1, wherein processor is further configured to, based on a determination that drift persists more than a threshold number of consecutive frames, apply to the image sensor a request to a brightness normalization.

6. The system of claim 1, wherein processor is further configured to, based on a determination that a drift persists more than a threshold number of consecutive frames, re-align a position of the extracted facial landmark estimates with reference points.

7. The system of claim 1, wherein processor is further configured to:
generate an average segment height of the user based on the time-sequenced matrix, and
recommend prescription related measurements based on the average segment height.

8. The system of claim 1, wherein the processor is further configured to:
perform ocular center placement tracking based on the time-sequenced matrix, and
recommend prescription related measurements based on the ocular center placement.

9. The system of claim 1, wherein the processor is further configured to:
pre-train the facial landmark detection MLM using a facial landmark training dataset comprising at least one of:
(a) facial images annotated with a distance between an outer or inner corners of both eyes,
(b) facial images annotated with left and right boundaries of each iris,
(c) facial images annotated with a pupil center for each eye,
(d) facial images annotated with landmarks at a widest visible part of the face,
(e) facial images annotated with a nasion point or a bottom of a nose bridge, and
(f) ground truth labels for the facial images to serve as a target output for the facial landmark detection MLM.

10. The system of claim 1, wherein the processor is further being configured to:
pre-train the self-correction MLM using a self-correction training dataset comprising at least one of:
(a) time-sequenced video frame data comprising a plurality of consecutive video frames, each frame labeled and annotated with ground truth facial landmark estimates to enable tracking of landmark positions across time,
(b) temporally annotated datasets comprising facial landmark annotations with frame-to-frame smoothness indicators or velocity profiles to identify inconsistencies and abrupt deviations in facial landmark movement,
(c) synthetically altered datasets comprising facial landmark annotations with artificial jitter, occlusion, or landmark drift introduced into otherwise smooth facial landmark sequences, paired with original ground truth landmarks as target outputs for self-correction training,
(d) spatial and temporal landmark representations comprising structured encodings of facial geometry and frame-delta information to contextualize facial landmark changes across consecutive frames,
(e) multimodal datasets comprising facial imagery, visual embeddings, head pose data, and synchronized audio or speech streams to support correlation between audio-visual cues and facial landmark dynamics, or
(f) ground truth labels for the time-sequenced video frame data, temporally annotated datasets, synthetically altered datasets, spatial and temporal landmark representations, and multimodal datasets to serve as a target output for the self-correction MLM.

11. A method for correcting facial landmark positions, comprising:
performing a facial scan by capturing an image of a face of a user and generating a plurality of video frames from the captured image;
extracting facial landmark estimates corresponding to the face of the user from each of the plurality of video frames using a pre-trained facial landmark detection machine learning model (MLM) configured to identify anatomical features of the face of the user, wherein the extracted facial landmark estimates comprise at least one of: an eye estimate, a pupil estimate, or face width;
storing each of the extracted facial landmark estimates as a vector in a time-sequenced matrix;
comparing consecutive video frames from the plurality of video frames to detect facial landmark drift using a pre-trained self-correction MLM;
recalibrating the extracted facial landmark estimates exhibiting drift using the pre-trained self-correction MLM; and
recommending eyewear frames based on the extracted facial landmark estimates that do not exhibit drift and the recalibrated facial landmark estimates.

12. The method of claim 11, wherein the pre-trained self-correction MLM is further configured to perform the recalibration on the extracted facial landmark estimates exhibiting drift by:
applying a temporal smoothing function on a plurality of the extracted facial landmark estimates to recalibrate the extracted facial landmark estimates using a Kalman filter,
generating confidence scores for the recalibrated facial landmark estimates based on at least one of: sharpness, consistency across video frames, clarity, symmetry and deviations from expected positions, and
based on a determination that a confidence score for any one of the recalibrated facial landmark estimates falls below a predefined threshold, provide to an image sensor a rescan request.

13. The method of claim 11, further comprising: based on a determination that drift persists more than a threshold number of consecutive frames, providing to an image sensor a rescan request.

14. The method of claim 13, further comprising: providing to the image sensor a rescan request by sending to a display a message to re-adjust a facial angle of the user.

15. The method of claim 11, further comprising: based on a determination that drift persists more than a threshold number of consecutive frames, applying to an image sensor a request to a brightness normalization.

16. The method of claim 11, further comprising: based on a determination that a drift persists more than a threshold number of consecutive frames, re-align a position of the extracted facial landmark estimates with reference points.

17. The method of claim 11, further comprising:
generating an average segment height of the user based on the time-sequenced matrix, and
recommending prescription related measurements based on the average segment height.

18. The method of claim 11, further comprising:
performing ocular center placement tracking based on the time-sequenced matrix, and
recommending prescription related measurements based on the ocular center placement.

19. The method of claim 11, further comprising:
pre-training the facial landmark detection MLM using a facial landmark training dataset comprising at least one of:
(a) facial images annotated with a distance between an outer or inner corners of both eyes,
(b) facial images annotated with left and right boundaries of each iris,
(c) facial images annotated with a pupil center for each eye,
(d) facial images annotated with landmarks at a widest visible part of the face,
(e) facial images annotated with a nasion point or a bottom of a nose bridge, and
(f) ground truth labels for the facial images to serve as a target output for the facial landmark detection MLM.

20. The method of claim 11, further comprising:
pre-training the self-correction MLM using a self-correction training dataset comprising at least one of:
(a) time-sequenced video frame data comprising a plurality of consecutive video frames, each frame labeled and annotated with ground truth facial landmark estimates to enable tracking of landmark positions across time,
(b) temporally annotated datasets comprising facial landmark annotations with frame-to-frame smoothness indicators or velocity profiles to identify inconsistencies and abrupt deviations in facial landmark movement,
(c) synthetically altered datasets comprising facial landmark annotations with artificial jitter, occlusion, or landmark drift introduced into otherwise smooth facial landmark sequences, paired with original ground truth landmarks as target outputs for self-correction training,
(d) spatial and temporal landmark representations comprising structured encodings of facial geometry and frame-delta information to contextualize facial landmark changes across consecutive frames,
(e) multimodal datasets comprising facial imagery, visual embeddings, head pose data, and synchronized audio or speech streams to support correlation between audio-visual cues and facial landmark dynamics, or
(f) ground truth labels for the time-sequenced video frame data, temporally annotated datasets, synthetically altered datasets, spatial and temporal landmark representations, and multimodal datasets to serve as a target output for the self-correction MLM.
